(19) United States
(12) Patent Application Publication
(10) Pub. No.: US 2009/0220503 A1
(43) Pub. Date: Sep. 3, 2009

(54) METHOD FOR TREATING CANCERS WITH INCREASED RAS SIGNALING

(75) Inventor: Douglas V. Faller, Weston, MA (US)

Correspondence Address: RONALD I. EISENSTEIN, 100 SUMMER STREET, NIXON PEABODY LLP, BOSTON, MA, 02110, US

(73) Assignee: THE TRUSTEES OF BOSTON UNIVERSITY, Boston, MA (US)

(21) Appl. No.: 12/282,432

(22) PCT Filed: Mar. 12, 2007
(86) PCT No.: PCT/US2007/006174

§ 371 Date: Mar. 20, 2009

Related U.S. Application Data
(60) Provisional application No. 60/780,970, filed on Mar. 10, 2006.

(51) Int. Cl. A61K 39/395; A61P 35/00; A61K 31/713; A61K 31/335; A61K 31/445; A61K 31/55

(52) U.S. Cl. 424/133.1; 514/44 A; 514/450; 514/315; 514/217.11

(57) ABSTRACT

Disclosed herein are methods for treating a subject with, or at risk for, developing a tumor which has aberrantly increased Ras signaling. The method involves obtaining a biological sample from the subject, determining whether the biological sample contains cells which have aberrantly increased Ras signaling, and administering an agent that selectively inhibits Protein Kinase C (PKC) delta to the subject upon determination of the aberrantly increased Ras signaling, to thereby inhibit PKC-delta in the cell. The increased Ras signaling may result from expression of activated Ras, e.g. resulting from mutations in codon 12, 13, 59, 61, 63, 116, 117, or 146. Such mutations can be determined by detection of a nucleotide sequence encoding an activated form of Ras protein, or by detection of the activated Ras protein. The increased Ras signaling may result from over-expression of wild-type Ras, over-activation of wild-type Ras, or increased activation of one or more effector pathways downstream of Ras. The tumor cells of the individual may be malignant or non-malignant. The inhibitor(s) of PKC-delta may inhibit PKC-delta gene expression, reduce PKC-delta protein levels, and/or inhibit PKC-delta protein function by inhibiting kinase activity. Appropriate inhibitors are Rottlerin, Balanol, balanol analogs, KAI 9S03, and combinations thereof. Also disclosed are methods for determining the likelihood of effectiveness of administering an agent that selectively inhibits PKC-delta to a subject with a tumor. The methods involve determining the presence or absence of aberrantly increased Ras signaling in the tumor, wherein the presence of aberrantly increased Ras signaling indicates that administration of the PKC-delta inhibitor is likely to be effective.

### RELATED APPLICATIONS

This application claims benefit under 35 USC §119(e) of U.S. Ser. No. 60/780,970 filed on Mar. 10, 2006.

### GOVERNMENT SUPPORT

This invention was supported, in part, by National Institutes of Health (NIH) Grant No. CA112102. The government of the United States has certain rights to the invention.

### FIELD OF THE INVENTION

The present invention is related generally to the field of cancer biology. In particular, the invention relates to compositions and methods of treatment for individuals having cancers with over-activation of the Ras signaling pathway.

### BACKGROUND OF THE INVENTION

The Ras oncogene family is among the most commonly mutated group of genes in human cancer. p21^{Ras} proteins have been shown to influence proliferation, differentiation, transformation, and apoptosis by relaying mitogenic and growth signals from the membrane into the cytoplasm and the nucleus.

Its protein products, p21^{ras}, code for three closely-related, small proteins including H-ras, K-ras and N-ras. p21^{Ras} proteins are localized in the inner plasma membrane, bind GDP and GTP and possess an intrinsic GTPase activity. p21^{Ras} proteins function as plasma membrane-bound guanine nucleotide binding proteins and act as molecular switches, thereby regulating signal transduction pathways for hormones, growth factors and cytokine receptors (Wiesmuller L, 1994). Several downstream effector proteins of p21^{Ras} have been identified which bind preferentially to p21^{Ras} in the GTP-bound state, including Raf, phosphoinositide 3-OH kinase (PI₃K), and a family of GDP-GTP exchange factors for the Ral small GTPases (Ral-GDS). These effectors in turn contribute to activation of the Raf1/MAPK pathway, the PI3K pathway, and the RasGDS/Ras/Rho pathway, respectively. Raf proteins, which are proto-oncogene-encoded serine/threonine kinases, activate the Raf1/MAPK signaling pathway. PI₃K activation results in the activation of the anti-apoptotic serine/threonine kinase Akt, among other molecules. Other p21^{Ras} targets include the GTPase-activating proteins, p120GAP and neurofibrin (Adiei A A, 2001). Aberrant signaling through p21^{Ras} pathways occurs as a result of several different classes of mutational damage in tumor cells.

Mutations of the p21^{Ras} genes themselves are common in human tumors. Some 20% of human tumors have activating point mutations in one of the three p21^{Ras} genes, most frequently in Ki-Ras (about 85% of total), followed by N-Ras (about 15%), then Ha-Ras (less than 1%). A particularly high incidence of Ras gene mutations has been reported in malignant tumors of the pancreas (80-90%, K-ras), in colorectal carcinomas (30-60%, K-ras), in non-melanoma skin cancer (30-50%, H-ras), and in hematopoietic neoplasia of myeloid origin (18-30%, K- and N-ras) (Downward J, 2003). Activating Ras mutations all compromise the GTPase activity of p21^{Ras}, preventing GAPs (GTPase Activating Proteins) from promoting hydrolysis of GTP on p21^{Ras}, and therefore causing p21^{Ras} to accumulate in the active (GTP-bound) form. Most mutational activation of p21^{Ras} in tumors is accounted for by mutations in codons 12, 13 and 61 (Bos J L. Cancer Res. 49:4682-9 (1989)). However, activating mutations of the Ras protein can arise from specific point mutations localized in codons 12, 13, 59, 61, 63, 116, 117, and 146, all of which lock p21^{Ras} protein in the active GTP-bound state and stimulate the downstream signaling cascades in the absence of extrinsic p21^{Ras} activation.

In addition to its central involvement in cell proliferation, recent studies indicate that the presence of an activated p21^{Ras} protein sensitizes transformed or malignant cells to apoptotic stimuli (Thimmaiah K N, 2003; Shao J, 2000; Liou J S, 2004; Liou J S, 2000; Chen C Y, 1996). Various signaling pathways may be involved in this anti-apoptotic activity. Previous studies demonstrated overall suppression of protein kinase C (PKC) activity in cells expressing activated p21^{Ras} rapidly induces apoptosis via FADD/caspase-8 signaling (Chen C Y, 1996). Reactive oxygen species have also been shown necessary as downstream effectors of the Ras-mediated apoptotic response to PKC inhibition (Liou J S, 2000).

There are at least 12 PKC isoforms that are classified into three subfamilies according to the structure of the N-terminal regulatory domain, which determines their sensitivity to the second messengers Ca²⁺ and diacylglycerol (DAG) (Parker P J, 2004). Despite the high degree of homology, however, there is a surprising degree of non-redundancy. Thus, individual PKC isoforms mediate different and unique cellular functions in different cell types and different tissues (Jaken S, 2000). PKCδ belongs to the subfamily of novel isoforms (PKCδ, PKCε, PKCθ and PKCη), which are insensitive to Ca²⁺. It is widely regarded as having pro-apoptotic properties (Basu A 2003; Brodie, 2003). Caspase activation mediates cleavage of PKCδ which results in release of the active catalytic domain (Ghayur T, 1996). In addition, PKCδ activity is known to initiate a number of pro-apoptotic signals, such as increased expression and stability of p53 (Johnson C L, 2002 and Abbas T, 2004), mitochondrial cytochrome C release (Majumder P K, 2000 and Basu A, 2001) and c-Ab1 activation (Sun X, 2000). Under certain conditions however, PKC-delta has been reported to have a protective role in cell survival. PKCδ has been reported to regulate B-lymphocyte survival (Mechlenbrauker I, 2004). Knock-out experiments have shown that PKCδ-deficient mice have a severely deregulated immune system and develop autoimmune disease (Mechlenbrauker I, 2002 and Miyamoto A, 2002). Thus, the role of PKCδ activation regarding apoptosis remains incompletely elucidated.

### SUMMARY OF THE INVENTION

Aspects of the present invention relate to methods for treating a subject with a proliferative disorder, herein referred to as a tumor. The method comprises determining the Ras genotype of the tumor, that is, looking for the presence of increased Ras signaling. A subject having a tumor associated with increased Ras signaling in the tumor is administered a PKC-delta targeting treatment. Preferably, the subject is administered a PKC-delta targeting treatment that selectively targets PKC-delta over other PKC isoforms.

Other aspects of the present invention relate to methods for directing treatment of a subject with a tumor. The status of the level of Ras signaling of the subject's tumor indicates the direction of treatment. A subject is directed toward a PKC-delta targeting treatment where the Ras signaling of the subject's tumor is increased relative to comparable cells.

Another aspect of the present invention relates to a method for determining the likelihood of effectiveness of a PKC-delta targeting treatment in a subject having tumor. The presence of increased Ras signaling in the subject's tumor indicates that administering a PKC-delta targeting treatment is likely to be effective.

One aspect of the present invention relates to a method for treating a subject with, or at risk for, developing a tumor which has aberrantly increased Ras signaling, comprising obtaining a biological sample from the subject; determining whether the biological sample contains cells which have aberrantly increased Ras signaling; and administering an agent that selectively inhibits Protein Kinase C (PKC) delta to the subject upon determination of the aberrantly increased Ras signaling, to thereby inhibit PKC-delta in the cell. In one embodiment, the aberrantly increased Ras signaling results from one or more occurrences, including expression of activated Ras, over-expression of wild-type Ras, or over-activation of wild-type Ras. Expression of activated Ras may be detected by ELISA, western blot, antibody staining, immunohistochemistry, immunofluorescence, or any combination thereof. Alternatively, it may be detected by determination of the presence of a mutation in a Ras nucleic acid sequence, by polymerase chain reaction, primer-extension, allele-specific probe hybridization, allele-specific primer extension, allele-specific amplification, nucleotide sequencing, 5′ nuclease digestion, molecular beacon assay, oligonucleotide ligation assay, single-stranded conformation polymorphism, or combinations thereof.

Another aspect of the present invention relates to a method for determining the likelihood of effectiveness of administering an agent that selectively inhibits PKC delta to a subject with a tumor comprising determining the presence or absence of aberrantly increased Ras signaling in the tumor, wherein the presence of aberrantly increased Ras signaling indicates that administration of the PKC delta inhibitor is likely to be effective.

### BRIEF DESCRIPTION OF THE DRAWINGS

FIGS. 1A-1C show effects of different PKC isoform inhibitors on proliferation of mouse fibroblast cells and human pancreatic cells. Cells were grown to 80% confluence in 96-well plates, then treated with the inhibitors at the concentrations indicated in Table I. The corresponding solvents with equivalent volume were used as vehicle controls. After 48 h treatment, cell proliferation was evaluated by MTT assay. Results are presented as the means ±SD. Statistical significance was determined using a paired Student's t-test, and p-values ≦0.05 were considered significant. FIG. 1A shows Hs766T and MIA PaCa-2 cells. FIG. 1B shows Balb and KBalb cells. FIG. 1C shows an assay of cellular p21^{Ras} activity. Nuclear-free lysates containing a total of 400 μg protein from each indicated cell type were used for analysis of Ras activity by Raf-RBD pull down. Equal loading was demonstrated by re-probing the blot with anti-actin Ab.

FIGS. 2A-2B show the effects of rottlerin on PKCδ activity and protein levels. Cells were treated with rottlerin (20 μM) for 48 h, then harvested for analysis. (FIG. 2A) Assay of PKCδ kinase activity. 200 μg of protein lysates were for immunoprecipitation. The means ±SD were obtained from at least three independent experiments, and actin antibody was used to obtain a background, which was subtracted. (FIG. 2B) PKCδ protein expression after rottlerin treatment. 40 μg aliquots of the same protein lysates were separated and immunoblotted using an anti-PKCδ monoclonal antibody.

FIGS. 3A-3D show the effect of PKCδ knockdown on viability of cells expressing activated p21^{Ras}. (FIG. 3A) Immunoblot analysis of PKCδ expression in NIH/3T3-Ras and MIA PaCa-2 cells transfected with PKCδ siRNA-1 and -2. A total of 50 nM of PKCδ siRNA was used and a scrambled siRNA was used as negative control. MIA PaCa-2 cells (FIG. 3B) and NIH/3T3-Ras cells (FIG. 3C) were transfected with pRNA-U6.1-GFP-control siRNA or pRNA-U6.1-GFP-PKCδ siRNA-2. After 72 h, apoptosis were examined by TUNEL reagent. Shown are 200× magnifications. (FIG. 3D) Quantitation of apoptotic cells in the transfected populations, with error bars indicating SD.

FIGS. 4A-4D show activation of apoptosis by individual p21^{Ras}-effector pathways. FIG. 4A shows NIH/3T3 cells stably-expressing an activated (V12) p21^{Ras} or p21^{Ras} effector loop-mutants were exposed to rottlerin (20 μM) for 60 h. Cells were fixed and stained with propidium iodide, and the apoptotic (hypodiploid) fractions were evaluated by PI staining and flow cytometric analysis using the FL2-H channel. Cells transfected with the empty pSG5 vector served as negative controls. FIG. 4B shows NIH/3T3 cell lines stably expressing the p21^{Ras} downstream effectors Raf-22W (Raf-1), P110-CAAXC (PI₃K), RIF-CAAX (RIF), or RalA-28N (as a negative control) were treated with rottlerin (20 μM) for 60 h and analyzed for apoptotic fraction. FIG. 4C shows suppression of apoptosis by inhibition of PI₃K activity. NIH/3T3 cells were pre-treated for 30 minutes with Ly294002 (10 μM) before rottlerin (20 μM) was added. The apoptotic (hypodiploid) fractions were evaluated 60 h later. FIG. 4D shows cells were pre-treated with vehicle solvent, LY294002 (10 μM) or MAPK inhibitor PD98995 (10 μM) and then treated with rottlerin (20 μM). The apoptotic (hypodiploid) fractions were evaluated 60 h later. Cells were fixed and stained with propidium iodide. DNA fragmentation was analyzed by flow cytometry using the FL2-H channel.

FIGS. 5A-5C show regulation of PKCδ levels by p21^{Ras}. FIG. 5A shows immunoblot analysis of PKCδ expression in matched cell lines pairs NIH/3T3 and NIH/3T3-Ras; Balb and Kbalb; and human pancreatic epithelial cell tumor lines BxPC-3 and MIA PaCa-2. Blots were stripped and reprobed for p21^{Ras} and β-actin. FIG. 5B shows NIH/3T3 cells were transfected with the pSG5-H-ras vector and harvested at the time points indicated. The NIH/3T3-Ras cell line was used as positive control. FIG. 5C shows effects of a p21^{Ras} inhibitor and a PI₃K inhibitor on PKCδ expression. NIH/3T3-Ras cells were treated with FPT inhibitor III (100 μM) or with Ly294002 (10 μM) for 48 h, then lysed and subjected to immunoblot assay for PKCδ. Blots were then stripped and reprobed for p21^{Ras} and β-actin.

FIGS. 6A-6B show PKCδ half-life in cells expressing activated p21^{Ras}. Balb and Kbalb cells at confluence were treated with cycloheximide (100 μg/ml) for the indicated length of time. Cells were lysed and equal protein samples (100 μg) were separated by SDS-PAGE and analyzed by immunoblotting. A representative blot is shown in FIG. 6A. Levels of PKCδ protein as a function of time, after normalization for loading by analysis of β-actin levels, are shown in FIG. 6B. Error bars represent the S.D. from five independent experiments.

FIGS. 7A-7B show the effect of isolated p21^{Ras} effector pathways on PKCδ expression. Immunoblots of PKCδ in: (FIG. 7A) NIH/3T3-Ras cells or NIH-3T3 cells stably-expressing the indicated Ras downstream effectors or empty vector as control (left panel) and KBalb cells or Balb cells stably-expressing the indicated Ras downstream effectors or empty vector as control (right panel); (FIG. 7B) cells stably-expressing Ras-effector loop mutants. A total of 50 μg of protein was separated on a 10% SDS-PAGE for each sample.

FIGS. 8A-8C show sequences for the three ras genes. FIG. 8A is the nucleic acid sequence of H-ras (GenBank Accession No. NM_{—}005343; SEQ ID NO: 1). FIG. 8B is the nucleic acid sequence of K-ras (GenBank Accession No. NM_{—}004985; SEQ ID NO: 2). FIG. 8C is the nucleic acid sequence of N-ras (GenBank Accession No. NM_{—}002524; SEQ ID NO: 3).

FIG. 9 is a schematic representation of the chemical structure of Rottlerin.

FIG. 10 is a schematic representation of the chemical structure of Balanol. The (−) enantiomer possesses the inhibitory activity.

### DETAILED DESCRIPTION OF THE INVENTION

Aspects of the present invention arise from the discovery that protein kinase C (PKC) delta activity is required for survival of cells with a proliferative disorder which possess aberrantly increased Ras signaling. The presence of increased Ras signaling (e.g. through expression of an activating p21^{Ras} mutation), either naturally-occurring or experimentally-induced, correlates with increased expression of PKC-delta mRNA transcript and protein. Conversely, inhibition of the aberrant Ras signaling correlates with decreased expression of PKC-delta. This increase in PKC-delta in the cell is required for the cell survival.

In one embodiment, activation of a downstream pathway such as Raf1/MAPK, RasGDS/Ras/Rho and PI3K will also confer apoptosis sensitivity to inhibition of PKC-delta in an abnormally proliferating cell such as a malignant cell. Such activation of the pathway(s) is intended to be encompassed by the term Ras signaling. These pathways can be activated by known means such as increased Ras signaling.

Accordingly, aspects of the present invention relate to treatment of a subject who is diagnosed with a proliferative disorder (non-malignant or malignant) which involves abnormally proliferating cells that have aberrantly increased Ras signaling. The increase in Ras signaling may result from one or more of several possible occurrences in the cell, including, without limitation, expression of activated Ras, over-expression of wild-type Ras, over-activation of Ras, and over-activation (e.g. constitutive activation) of one or more effector pathways downstream of Ras. Identification of aberrantly increased Ras signaling in the abnormally proliferating cells of a subject indicates that a PKC-delta targeting (inhibiting) treatment (such as a PKC-delta inhibitor) is likely to be effective against the abnormally proliferating cells, and therefore such therapy is likely to be effective in the treatment of the subject. In another embodiment, the presence of aberrantly increased Ras signaling in a subject's tumor directs treatment of the subject toward a PKC-delta targeting treatment.

The findings and methods disclosed herein are equally applicable to malignant (cancerous) disorders and to non-malignant proliferative disorders (e.g. a non-malignant tumor). Such abnormally proliferating cells, malignant and non-malignant, are herein referred to as a tumor or tumor cells. Malignant tumor cells are also referred to herein as cancer or cancer cells. Tumor cells (malignant or non-malignant) may exist as a solid tumor, or alternately may exist as isolated individual cells, e.g. overproduced immune cells as occur in leukemia, or loosely associated cells, or free floating cells. In addition, tumor cells or tumors may be located at more than one position in the body, e.g. from metastasis of malignant cells, free floatation, or independent arisal.

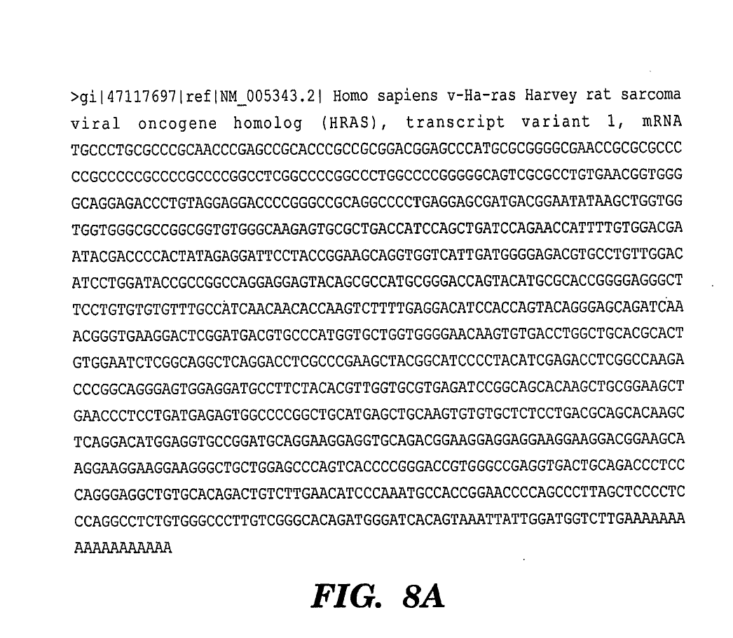

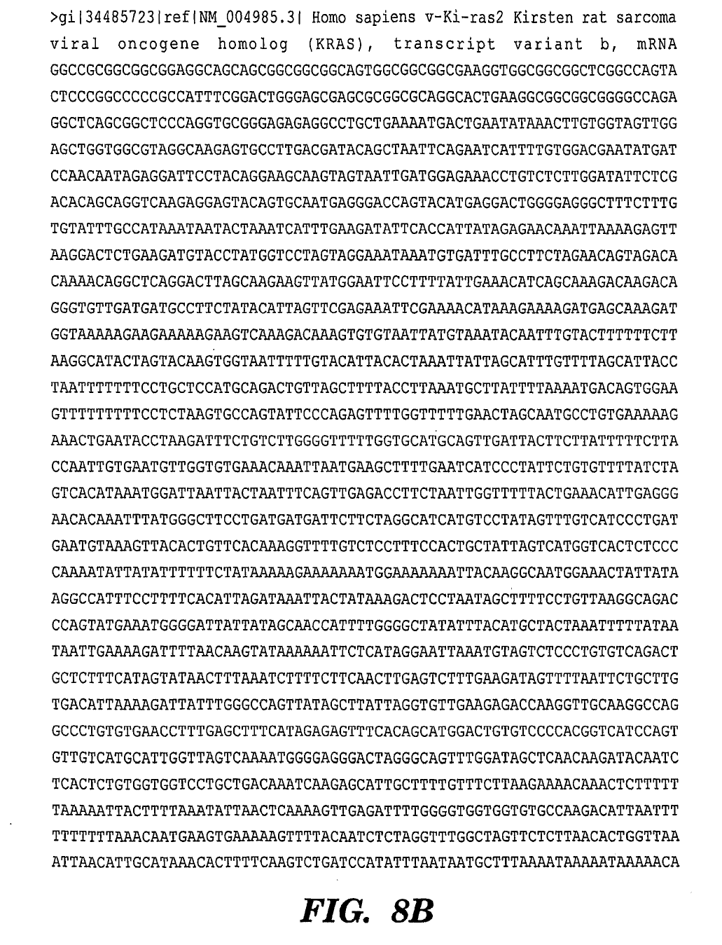

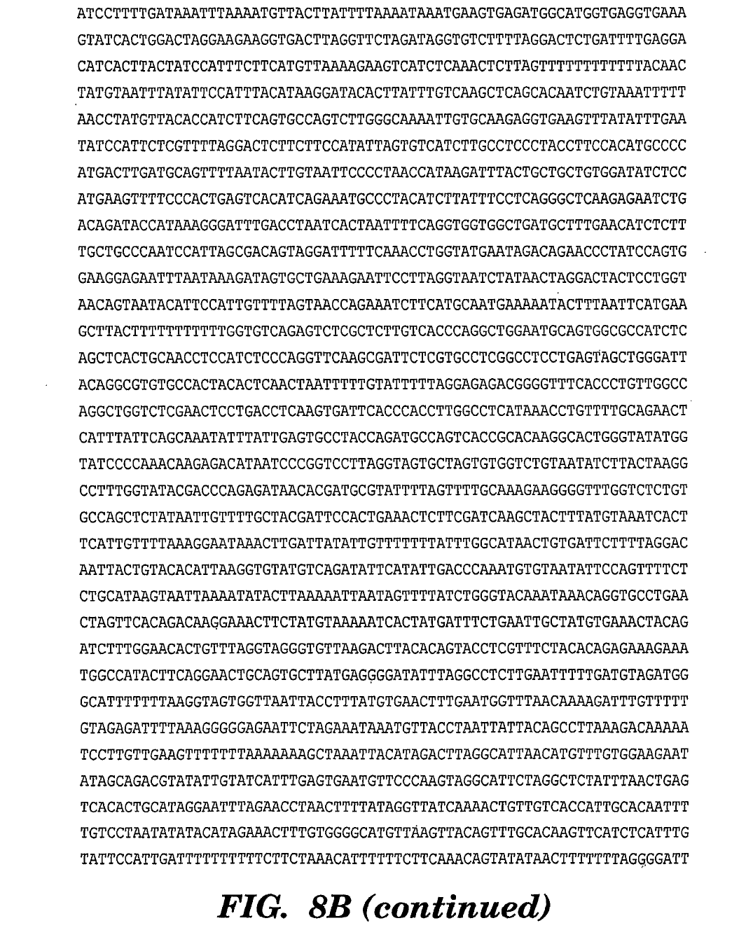

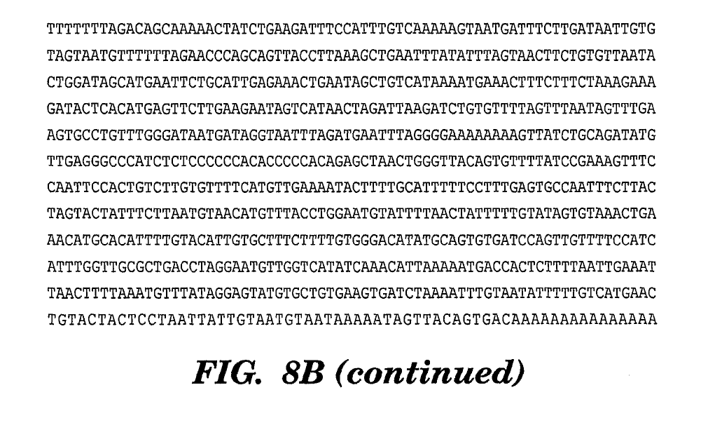

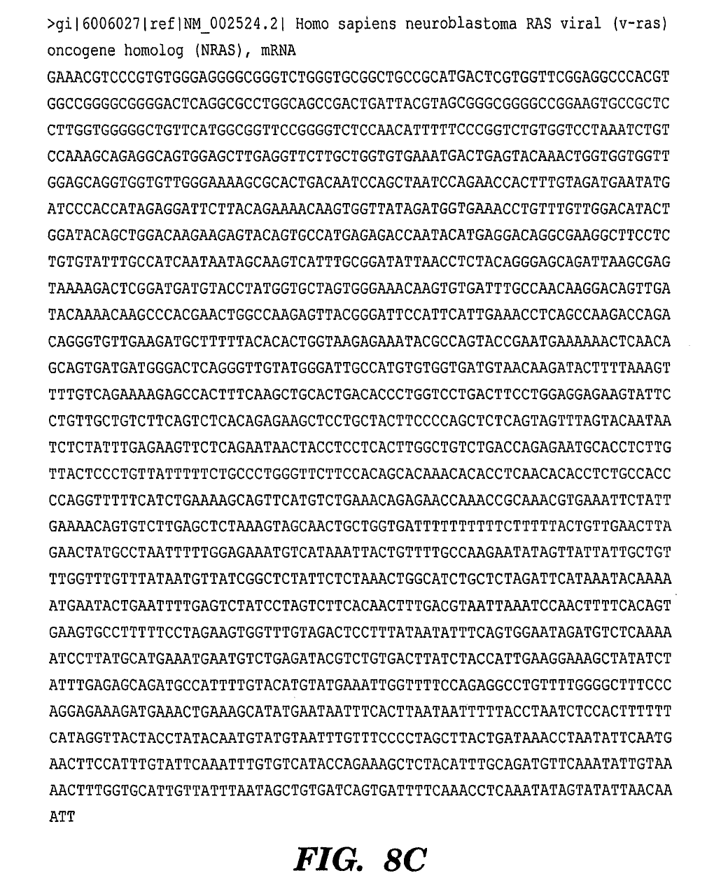

The terms “Ras” and “p21^{Ras}” are used interchangeably herein to refer to the protein product of a Ras gene. The mammalian ras gene family consists of the Harvey and Kirsten ras genes (c-Hras1 and c-kras2), an inactive pseudo gene of each (c-Hras2 and c-kras1) and the N-ras gene. The p21^{Ras} protein products of the three ras genes (p21^{H-Ras}, p21^{K-Ras}, and p21^{N-Ras}, respectively) differ significantly only in their C-terminal 40 amino acids, and each are activated by the same or corresponding activating mutations. The three ras gene sequences, as well as their protein products, for a variety of animals, (e.g. mammals, including humans) are well known in the field. Examples of each of the human ras gene coding sequences are provided in FIG. 8: H-ras in FIG. 8A, K-ras in FIG. 8B, and N-ras in FIG. 8C.

Aberrant signaling through p21^{Ras} pathways occurs as a result of several different classes of mutational damage in tumor cells. Table 1 (copied from Downward, 2003) provides a list of methods of increased activation of Ras signaling pathway in different tumors.

TABLE 1
Activation of RAS signalling pathways in different tumours

| Defect of mutation | Tumour type | Frequency (%) |
|---|---|---|
| RAS mutation | Pancreas | 90 (K) |
| | Lung adenocarcinoma | 35 (K) |
| | (non-small-cell) | |
| | Colorectal | 45 (K) |
| | Thyroid (Follicular) | 55 (H, K, N) |
| | Thyriod | 60 (H, K, N) |
| | (Undifferentiated | |
| | papilary) | |
| | Seminoma | 45 (K, N) |
| | Melanoma | 15 (N) |
| | Bladder | 10 (H) |
| | Liver | 30 (N) |
| | Kidney | 10 (H) |
| | Myelodysplastic syndrome | 40 (N, K) |
| | Acute myelogenous leukaemia | 30 (N) |
| BRAF mutation | Melanoma | 66 |
| | Colorectal | 12 |
| EGFR overexpression | Most carcinomas | >50 |
| ERBB2 amplification | Breast | 30 |
| PYEN loss | Glioblastoma multiforme | 20-30 |
| | Prostate | 20 |
| | Pancreas | 40 |
| AKT2 amplification | Ovarian | 12 |
| | Pancreas | 10 |
| PI3K amplification | Ovarian | 40 |

EGFR, epidermal-growth-factor receptor;
PI3K, phosphatidyinositol 3-kinase,
H, K and N refer to HRAS, KRAS and NRAS, respectively.

As used herein, the term “activated Ras mutation” refers to the presence of a genomic mutation in a ras gene which leads to the expression of an activated form of the Ras protein. The term “wild-type” is used herein to refer to nucleic acids encoding Ras proteins that do not contain activating mutations, and also to refer to Ras proteins which do not result from activating mutations.

A common mechanism of increased Ras signaling in a cell is through expression of an activated p21^{Ras} protein. As such, aspects of the present invention relate to a method of targeting tumor cells (carcinogenic or benign) which contain an activated p21^{Ras} protein, by selective suppression of PKC-delta activity and consequent induction of apoptosis. In one embodiment, the invention involves a method for determining the status of Ras in a subject's tumor, i.e., looking for the presence of an activated Ras mutation.

Wild-type Ras proteins, found in normal, healthy individuals, cycle between an active (GTP bound) state and an inactive (GDP bound) state. Activated Ras proteins result from activating mutations which have decreased inherent GTPase activity, and are resistant to the action of GTPase-activating proteins (GAPs), the natural negative regulators of Ras proteins. Thus, these mutations, e.g., mutations localized in codons 12, 13, 59, 61, 63, 116, 117, and 146, are activating mutations resulting in the Ras protein being locked in an active conformation. The presence of the activated form of Ras in a cell leads ultimately to inappropriate cell proliferation signaling.

Activated Ras proteins play a key role in the development of many human cancers. Such mutations in Ras are observed in approximately one third of all tumors (Bos, Cancer Res 49:4682-4689 [1989]; and Clark and Der, in GTPases in Biology [eds. Dickey and Birmbauer], Springer-Verlag London Ltd., pp. 259-287 [1993]). Indeed, the frequency of Ras mutation approaches 100% in some types of tumors (e.g., pancreatic adenocarcinoma). In addition to the correlation between the presence of activated Ras mutations in a high percentage of a variety of cancers, a wealth of experimental evidence indicates that increased Ras activity is involved in malignant cell transformation and tumorigenesis. For example, activated forms of the Ras protein can be used to experimentally transform cells in culture and induce tumors in animal models. Furthermore, deletion of the activated Ras gene from tumor cell lines impairs their tumorigenicity (Paterson et al., Cell 51:803-812 [1987]; and Shirasawa et al., Science 260:85-88 [1993]).

In addition to actual mutations in ras gene coding sequences, mutations which lead to over-expression of wild-type (non-activated) p21^{Ras} also contribute to tumor formation. Mutations in a cell which might cause over-expression include, without limitation, gene amplification of a ras gene, mutations in the ras promoter region, mutations in positive or negative regulators of ras mRNA or Ras protein expression. A variety of such mutations are known in the art and can be detected by the skilled practitioner.

Changes in a cell which lead to over-activation of wild-type Ras protein, e.g. mutations which result in over-expressed or overactive mutant forms of molecules upstream in Ras signal transduction, such as receptors that utilize Ras signaling, also contribute to tumor formation.

p21^{Ras} signaling pathways are commonly activated in tumors in which growth-factor-receptor tyrosine kinases have been over-expressed. The most common examples are EGF-R and ErbB2 (also known as HER2/neu), which are frequently activated by their over-expression in many types of cancer, including breast, ovarian and stomach carcinomas (Table 1) (Mendelsohn J & Baselga J. Oncogene 19:6550-65 (2000)). Similarly, a mutation in the EGF-R gene results in the expression of a truncated receptor that lacks part of the extracellular domain (Kuan C T, Wikstrand C J, & Bigner D D. Endocr. Relat Cancer 8:83-96 (2001)), and this mutated receptor is found to be over-activated in a significant proportion of glioblastomas and some other tumor types. EGF-R-family tyrosine kinases are also commonly activated by the autocrine production of EGF-like factors such as transforming growth factor-α (TGF-α) in tumors. The exact frequency of this activation is hard to establish in human tumors, but it seems to be very high in tumors of epithelial origin.

Previous findings combined with the findings presented herein establish that prolonged activation of endogenous wt-Ras, such as that by increased upstream signaling, is sufficient to make cells susceptible to apoptosis induced by PKC inhibition (Chen C Y, Liou J, Forman L W, & Faller D V. J. Biol. Chem. 273:16700-9 (1998)).

Mutations in a cell which decrease the negative regulation of Ras is a mechanism by which over-activation of wild-type Ras protein can occur in a tumor cell. One such possibility of this is GAP deletion. p21^{Ras} can also be activated in tumors by loss of Ras-GAPs (GTPase Activating Proteins). The NF1 protein is a Ras-GAP (GTPase-Activating Protein). The most significant known example is the loss of neurofibromin, which is encoded by the NF1 gene, (Weiss B, Bollag G, & Shannon K. Am. J. Med. Genet. 89:14-22 (1999)) which acts as a tumor suppressor. One allele is lost in people with type I neurofibromatosis—a dominant syndrome that is characterized by large numbers of benign and occasionally malignant, tumors in tissues of neural-crest origin. In the malignant tumors, both copies of NF1 have been lost, resulting in abnormal activation of p21^{Ras}.

Inactivation/deletion of NF1 or Ras-GAP is associated with hyperactive p21^{Ras} in tumor Cells, to the extent that inactivation of the negative regulators of Ras and somatic p21^{Ras} point mutations are functionally equivalent (Kalra et al., Blood 84:3435-9 (1994)). MPNST cell lines derived from patients with neurofibromatosis show decreased in vitro GAP activity and markedly elevated levels of Ras-GTP (Basu T N, Gutmann D H, Fletcher J A, Glover T W et al. Nature 356:713-(1992); DeClue J E, Papageorge A G, Fletcher J A, Diehl S R et al. Cell 69:265-73 (1992)). Increased Ras-GTP levels have also been reported in primary MPNSTs removed from patients with NF1 (Strober B E, Dunaief J L, Guha, & Goff S P. Mol. Cell Biol. 16:1576-83 (1996)) and in Schwann cells isolated from homozygous Nf1-deficient embryos (Kim H A, Rosenbaum T, Marchionni M A, Ratner N et al. Oncogene 11:325-35 (1995)). The results of the experiments presented in the Examples section herein, combined with various previous findings in the field, establish that downregulation of GAP is sufficient to render cells containing unmutated, wild-type p21^{Ras} susceptible to apoptosis induced by PKD inhibition (Chen C Y, Liou J, Forman L W, & Faller D V. J. Biol. Chem. 273:16700-9 (1998)).

Mutations which result in aberrantly increased activity of downstream effectors of Ras proteins also produce aberrantly increased “Ras” signaling in a tumor cell. One way this can occur is by mutation or amplification of p21Ras effectors (signal transduction molecules located downstream of Ras).

The Ras protein is known to activate at least three downstream pathways, including the Raf1/MAPK pathway, Ras GDS/Ras/Rho pathway, and PI₃K pathway. The results of experiments detailed in the Examples below indicate that activation of the PI₃K pathway is sufficient to confer apoptosis sensitivity to inhibition of PKC delta in a tumor cell. As such, treatment of a subject with a tumor which demonstrate increased activation of the PI₃K pathway is also encompassed in the embodiments of the invention. The methods of treatment herein can also be applied to treatment of such a tumor in an individual, following detection of the increased activity of the PI₃K pathway in cells of the tumor. The methods described herein can be applied to the determination of the activation, over-activation, or over-expression of the PI₃K pathway to determine if a subject should be administered a PKC-delta inhibitor to treat the subject's tumor. Activation of the PI₃K pathway can be determined, for example, by comparison of the activity of PI₃K in a tumor cell to the activity of PI₃K in a non-tumor cell of the same or similar origin. The activity of the PI₃K pathway can be determined and quantitated by a variety of methods known to the skilled practitioner, and applied to the method of the present invention. The methods of the present invention are intended to encompass all such methods of determining the activity of the PI₃K pathway.

Similarly, activation of the MAPK pathway has also been shown to significantly contribute to apoptosis sensitivity to inhibition of PKC delta in a tumor cell. As such, treatment of a subject with a tumor which demonstrates increased activation of the MAPK pathway is also encompassed in the embodiments of the invention. The methods of treatment herein can also be applied to treatment of such a tumor in an individual, following detection of the increased activity of the MAPK pathway in cells of the tumor. The methods described herein can be applied to the determination of the activation, over-activation, or over-expression of the MAPK pathway to determine if a subject should be administered a PKC-delta inhibitor to treat the subject's tumor. Activation of the MAPK pathway can be determined, for example, by comparison of the activity of MAPK, or other kinases within that pathway (e.g. Raf, MEK, MEKK) in a tumor cell to the activity of MAPK or other like kinase in a non-tumor cell of the same or similar origin. The activity of the MAPK pathway can be determined and quantitated by a variety of methods known to the skilled practitioner, and applied to the method of the present invention. The methods of the present invention are intended to encompass all such methods of determining the activity of the MAPK pathway.

Since activation of the PI₃K pathway has been shown sufficient to confer apoptosis sensitivity to PKC-delta inhibition in a tumor cell, and activation of the MAPK pathway also confers this apoptosis sensitivity, it may be useful to evaluate the activating Ras mutation present in the tumor to determine which downstream pathway(s) are activated, wherein the presence of an activated Ras mutant which has the ability to activate the PI₃K pathway and/or the MAPK pathway is especially indicative of the sensitivity of the tumor to PKC-delta inhibition.

As one example of over-activation (e.g. amplification) of a p21Ras effector is activation of Raf. B-Raf is frequently activated by mutation in human tumors (Table 1)—in particular, in melanomas (˜70%) and colon carcinoma (˜15%) (Davies H, Bignell G R, Cox C, Stephens P et al. Nature 417:949-54 (2002); Mercer K E & Pritchard C A. Biochim. Biophys. Acta 1653:25-40 (2003); Brose M S, Volpe P, Feldman M, Kumar M et al. Cancer Res. 62:6997-7000 (2002)). Mutations in B-Raf occur in a very limited number of residues in the kinase domain (with a single substitution [V599E] accounting for 80%), all of which result in kinase activation.

In addition, The PI₃K pathway is activated as a result of amplification of the p110α gene in a significant proportion of ovarian tumors, and/or by the amplification of its downstream target AKT2 in ovarian and breast tumors (Table 1) (Bellacosa A, de Feo D, Godwin A K, Bell D W et al. Int. J. Cancer 64:280-5 (1995). High levels of expression of AKT2 mRNA are observed in two ovarian carcinoma cell lines, OVCAR-3 and OVCAR-8. These lines exhibited ˜30-fold and ˜45-fold increases in AKT2 mRNA and protein levels, respectively, compared with A2780 ovarian carcinoma cell line, or cultured diploid human ovarian surface epithelial (HOSE) cells in early passage (Cheng J Q, Godwin A K, Bellacosa A, Taguchi T et al. Proc. Natl. Acad. Sci. U.S.A 89:9267-71 (1992)).

The most significant direct activation of this pathway in tumors comes from deletion of the tumor suppressor gene PTEN (phosphatase and tensin homologue). This gene encodes a lipid phosphatase that removes the phosphate from the 3′ position of PtdIns(3,4,5)P₃ and PtdIns(3,4)P₂, so reversing the accumulation of these second messengers that is caused by PI₃K. PTEN is deleted in ˜30-40% of human tumors (Table 1) (Simpson L & Parsons R. Exp. Cell Res. 264:29-41 (2001)), making it the second most significant tumor-suppressor gene after TP53 (which encodes p53 in humans). Results of experiments presented in the Examples section herein establish that activation either of the p21 Ras downstream effector pathways PI3K or Raf/MAPK is sufficient to render cells susceptible to apoptosis when PKC□ is inhibited, even in the presence of a normal, wild-type p21^{Ras}

Aberrant signaling pathways commonly found in tumors can result in prolonged or supra-physiological activation of endogenous, native, wild-type p21^{Ras} proteins. Constitutive, or ligand-independent, activation of growth factor receptors which act through p21^{Ras} is a major mechanism whereby p21^{Ras} can be activated to supra-physiological levels in the absence of activating mutations in p21^{Ras} itself.

Tumors which have over-expression of wild-type Ras protein, and also tumors which have over-activation of wild-type Ras protein, as well as tumors which express activated Ras proteins are expected to be susceptible to apoptosis brought about by selective inhibition of PKC-gamma. As such, the methods described herein can also be applied to detection of the over-expression or over-activation of the wild-type Ras protein in a tumor, with such detection being indicative of a likelihood of effectiveness of a PKC-delta targeting treatment in the treatment of a subject with the tumor. Once detected, the subject with the tumor would be administered a PKC-delta targeting treatment as described herein.

Detection of over expression of wild-type ras in a tumor can be performed routinely by quantitative comparison of levels of expressed ras mRNA or Ras protein to non-tumorigenic tissue of the same origin and even from the same individual. Increased wild-type ras mRNA or wild-type Ras protein in the tumor cells compared to the control cells indicates over-expression of the ras gene in the tumor. Over-activation, in the absence of over-expression can be determined, for instance, by measuring the percent of Ras in the GTP bound state in a cell. Comparison of the percent of GTP bound Ras of the tumor cells, to GTP bound Ras in similar non-tumor cells from the same individual is indicative of overactive Ras in a cell. Over-activation of Ras typically results from an aberrant increase activity of growth factor receptors which activate Ras, e.g. EGF-R and ErbB2.

Upon or prior to detection of aberrantly increased Ras signaling in a biological sample of the subject, it may be useful to identify or confirm over-activation (e.g. over-expression) of PKC-delta in the affected cells (e.g. tumor or pre-tumor) of the subject. Many methods of such determination of over-activity of a kinase molecule are known and can be adapted by the skilled practitioner to the detection of PKC-delta as described herein. For example, many of the methods described herein can be applied to the detection of PKC-delta activity and/or expression, and can be used to determine a corresponding increase in PKC-delta expression and/or activation. The skilled practitioner can further correlate a determined aberrant increased Ras signaling with an increase in PKC-delta expression and/or activity which is reported herein to confer apoptosis sensitivity to PKC-delta inhibition in the cells.

Protein kinase C (PKC) is a membrane-associated enzyme that is regulated by a number of factors, including membrane phospholipids, calcium, and membrane lipids such as diacylglycerols that are liberated in response to the activities of phospholipases (Bell et al. J. Biol. Chem. 1991. 266:4661-4664; Nishizuka, Science 1992. 258:607-614). The protein kinase C isozymes, alpha, beta, beta-1, beta-2 and gamma, require membrane phospholipid, calcium and diacylglycerol/phorbol esters for full activation. The delta, epsilon, eta, and theta forms of PKC are calcium-independent in their mode of activation. The zeta and lambda forms of PKC are independent of both calcium and diacylglycerol and are believed to require only membrane phospholipid for their activation. PKC- and isozyme-specific (e.g., PKC delta specific) modulators are described, e.g., in Goekjian et al. Current Medicinal Chemistry, 1999, 6:877-903; Way et al., Trends Pharmacol Sci, 2000, 21:181-7, and in U.S. Pat. No. 5,843,935.

Aberrant increased Ras signaling in cells of a tumor which results from one or more of a variety of mechanisms, can be determined by the skilled practitioner by a variety of methods. One method of determination is through comparison of Ras signaling in cells of a tumor to Ras signaling of comparable non-tumor cells. Additional methods of determination of increased Ras signaling are available, many of which detect one or more specific mechanism of increased Ras signaling in a cell. [Move down]

### Methods to Detect Ras Abnormalities

In one embodiment, the invention involves methods to determine the susceptibility of the tumor to PKC-delta inhibitors, preferably PKC-delta selective inhibitors. The methods involve determination of the ras genotype of a tumor in a subject. Such determination can be done by detection or identification of the presence of activating mutations in one or more of the ras genes, e.g., N-ras, H-ras, K-ras. Such mutations are known to commonly occur at codons 12, 13, 59, 61, 63, 116, 117, and 146 of H-ras, and the respective codons of other ras genes as well. Such a mutation, herein referred to as an activated Ras mutation, can, for example, be detected a mutation at the genomic level or by detecting the activated form of the Ras protein in the cell. The skilled practitioner will be aware of all activating Ras mutations known at the time of the analysis, and able to apply the technology known at that time to detect such mutations. Detection of an activated Ras mutation in a cancer cell is indicative of the susceptibility of the cancer cell to PKC-delta inhibitors, preferably PKC-delta selective inhibitors. A variety of methods of determining a genotype of a cell, e.g. a ras genotype, are well known to the skilled artisan. Several examples of such methods useful for determining the ras genotype of a cell (e.g. a tumor and/or cancer cell) are described herein. These examples are intended to be non-limiting. Any method of determination of a genotype of a cell, known at the time of the analysis, can be used to determine the ras genotype and therefore applied to the methods of the invention described herein.

The ras genotype is detected in a biological sample obtained from the subject. The biological sample may be a tumor biopsy. The biological sample may be cells obtained from a blood sample from the subject, e.g., a blood sample comprising tumor cells, e.g., circulating tumor cells. The biological sample may be obtained during surgical resection of a tumor. Nucleic acids, DNA or RNA, e.g., total RNA, mRNA, may be isolated or extracted from the biological sample. Protein may be extracted from the biological sample. Extracted or isolated nucleic acid or protein material may be used for detection of gene mutations, including gene expression. In one preferred embodiment, activated Ras mutations are detected in the biological sample, e.g. a biopsy, itself.

### Nucleic Acid Based Methods

Nucleic acid samples, including both DNA and RNA, can be genotyped to determine which allele(s) is/are present at any given genetic region, e.g., ras mutation position, of interest by methods well known in the art. The neighboring sequence can be used to design ras mutation detection reagents such as oligonucleotide probes, which may optionally be implemented in a kit format. Common mutation genotyping methods include, but are not limited to, TaqMan assays, molecular beacon assays, nucleic acid arrays, allele-specific primer extension, PCR-RFLP, allele-specific PCR, single-molecule dilution (SMD) (Ruano et al. (1990) Proc. Natl. Acad. Sci. USA 87:6296-6300), and coupled amplification and sequencing (CAS) (Ruano and Kidd (1991) Nucleic Acids Res. 19:6877-6882), arrayed primer extension, homogeneous primer extension assays, primer extension with detection by mass spectrometry, pyrosequencing, multiplex primer extension sorted on genetic arrays, ligation with rolling circle amplification, homogeneous ligation, OLA (oligonucleotide ligation assay; U.S. Pat. No. 4,988,167), multiplex ligation reaction sorted on genetic arrays, restriction-fragment length polymorphism, single base extension-tag assays, and the INVADER® assay (Third Wave Technologies, Inc., Madison Wis.). RT-PCR and other RNA specific amplification based methods may also be used. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrophoretic detection.

Various methods for detecting polymorphisms in ras coding sequences include, but are not limited to, methods in which protection from cleavage agents is used to detect mismatched bases in DNA duplexes (Myers et al., Science 230:1242 (1985); Cotton et al., PNAS 85:4397 (1988); and Saleeba et al., Meth. Enzymol. 217:286-295 (1992)), comparison of the electrophoretic mobility of variant and wild type nucleic acid molecules (Orita et al., PNAS 86:2766 (1989); Cotton et al., Mutat. Res. 285:125-144 (1993); and Hayashi et al., Genet. Anal. Tech. Appl. 9:73-79 (1992)), and assaying the movement of polymorphic or wild-type fragments in polyacrylamide gels containing a gradient of denaturant using denaturing gradient gel electrophoresis (DGGE) (Myers et al., Nature 313:495 (1985)). Sequence variations at specific locations of the ras nucleic acids can also be assessed by nuclease protection assays such as RNase and S1 protection or chemical cleavage methods.

In one method of PCR-based assay, an allele-specific primer hybridizes to a region on a target ras nucleic acid molecule that overlaps the mutation position, e.g., point mutations corresponding to codons 12, 13, 59, 61, 63, 116, 117, and 146 of a Ras gene, and only primes amplification of an allelic form to which the primer exhibits perfect complementarity (Gibbs, 1989, Nucleic Acid Res. 17 2427-2448). Typically, the primer's 3′-most nucleotide is aligned with and complementary to the mutation position of the target nucleic acid molecule. This primer is used in conjunction with a second primer that hybridizes at a distal site. Amplification proceeds from the two primers, producing a detectable product that indicates which allelic form is present in the test sample. A control is usually performed with a second pair of primers, one of which shows a single base mismatch at the polymorphic site and the other of which exhibits perfect complementarity to a distal site. The single-base mismatch prevents amplification or substantially reduces amplification efficiency, so that either no detectable product is formed or it is formed in lower amounts or at a slower pace. The method generally works most effectively when the mismatch is at the 3′-most position of the oligonucleotide (i.e., the 3′-most position of the oligonucleotide aligns with the target mutation position) because this position is most destabilizing to elongation from the primer (see, e.g., WO 93/22456). This PCR-based assay can be utilized as part of the TaqMan assay.

Another method suitable for the detection of activated Ras mutations, makes use of the 5′-exonuclease activity of a DNA polymerase to generate a signal by digesting a probe molecule to release a fluorescently labeled nucleotide. This assay is frequently referred to as a TaqMan assay (see, e.g., Arnold, et al., BioTechniques 25(1):98-106 (1998); and Becker, et al., Hum. Gene Ther. 10:2559-66 (1999) and U.S. Pat. Nos. 5,210,015 and 5,538,848). The TaqMan assay detects the accumulation of a specific amplified ras product during PCR. The TaqMan assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye may be at the 5′ most and the 3′ most ends, respectively, or vice versa. Alternatively, the reporter dye may be at the 5′ or 3′ most end while the quencher dye is attached to an internal nucleotide, or vice versa. In yet another embodiment, both the reporter and the quencher may be attached to internal nucleotides at a distance from each other such that fluorescence of the reporter is reduced.

During PCR, the 5′ nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target ras mutation-containing template which is amplified during PCR, and the probe is designed to hybridize to the target mutation site only if a particular mutation allele of ras is present.

Preferred TaqMan primer and probe sequences can readily be determined using the mutation and associated nucleic acid sequence information provided herein. A number of computer programs, such as Primer Express (Applied Biosystems, Foster City, Calif.), can be used to rapidly obtain optimal primer/probe sets. It will be apparent to one of skill in the art that such primers and probes for detecting the ras mutations are useful in diagnostic assays for determination of activated Ras mutations in tumors, and can be readily incorporated into a kit format. The present invention also includes modifications of the Taqman assay well known in the art such as the use of Molecular Beacon probes (U.S. Pat. Nos. 5,118,801 and 5,312,728) and other variant formats (U.S. Pat. Nos. 5,866,336 and 6,117,635).

Another method suitable for the detection of mutations in ras genomic DNA (H-ras, K-ras and/or N-ras) in a sample without amplification is the INVADER® technology available from Third Wave Technologies, Inc., Madison, Wis. Detailed protocols to use this technology to detect mutations may be found, e.g., in Hessner, et al., Clinical Chemistry 46(8):1051-56 (2000); Hall, et al., PNAS 97(15):8272-77 (2000); Agarwal, et al., Diag. Molec. Path. 9(3):158-64 (2000); and Cooksey, et al., Antimicrobial and Chemotherapy 44(5):1296-1301 (2000). In the Invader process, two short DNA probes hybridize to a ras target nucleic acid to form a structure recognized by a nuclease enzyme. For analysis of mutations such as single nucleotide mutations, two separate reactions are run—one for each gene variant, e.g. one for the activated Ras allele (H, K, or N) and one for the respective wild-type allele. If one of the probes is complementary to the sequence, the nuclease will cleave it to release a short DNA fragment termed a “flap”. The flap binds to a fluorescently-labeled probe and forms another structure recognized by a nuclease enzyme. When the enzyme cleaves the labeled probe, the probe emits a detectable fluorescence signal thereby indicating which activated Ras mutational variant is present.

Another method for detecting mutations, e.g., activated Ras mutations, useful in the methods of the invention is rolling circle amplification, which utilizes an oligonucleotide complementary to a circular DNA template to produce an amplified signal (see, for example, Lizardi, et al., Nature Genetics 19(3):225-32 (1998); and Zhong, et al., PNAS 98(7):3940-45 (2001)). Extension of the oligonucleotide results in the production of multiple copies of the circular template in a long concatemer. Typically, detectable labels are incorporated into the extended oligonucleotide during the extension reaction. The extension reaction can be allowed to proceed until a detectable amount of extension product is synthesized.

Another method for detecting activated ras mutations, is the use of two oligonucleotide probes in an OLA (see, e.g., U.S. Pat. No.4,988,617). In this method, one probe hybridizes to a segment of a target nucleic acid with its 3′ most end aligned with the activated Ras mutation site. A second probe hybridizes to an adjacent segment of the target nucleic acid molecule directly 3′ to the first probe. The two juxtaposed probes hybridize to the target ras nucleic acid molecule, and are ligated in the presence of a linking agent such as a ligase if there is perfect complementarity between the 3′ most nucleotide of the first probe with the mutation site. If there is a mismatch, ligation would not occur. After the reaction, the ligated probes are separated from the target nucleic acid molecule, and detected as indicators of the presence of a mutation, e.g., an activated Ras mutation.

The following patents, patent applications, and published international patent applications, which are all herein incorporated by reference in their entirety, provide additional information pertaining to techniques for carrying out various types of OLA: U.S. Pat. Nos. 6,027,889, 6,268,148, 5,494,810, 5,830,711, and 6,054,564 describe OLA strategies for performing SNP, e.g., mutation, detection; WO 97/31256 and WO 00/56927 describe OLA strategies for performing SNP detection using universal arrays, wherein a zipcode sequence can be introduced into one of the hybridization probes, and the resulting product, or amplified product, hybridized to a universal zip code array; WO 01/92579 (and Ser. No. 09/584,905) describes OLA or LDR (ligase detection reaction) followed by PCR, wherein zipcodes are incorporated into OLA probes, and amplified PCR products are determined by electrophoretic or universal zipcode array readout; U.S. application U.S. Pat. App. No. 20050053957 and WO 04/046343 describe SNPlex methods and software for multiplexed SNP detection using OLA followed by PCR, wherein zipcodes are incorporated into OLA probes, and amplified PCR products are hybridized with a zipchute reagent, and the identity of the single nucleotide mutation determined from electrophoretic readout of the zipchute. In some embodiments, OLA is carried out prior to PCR (or another method of nucleic acid amplification). In other embodiments, PCR (or another method of nucleic acid amplification) is carried out prior to OLA.

Another method for detecting mutations, e.g., activated Ras mutations, is based on mass spectrometry. Mass spectrometry takes advantage of the unique mass of each of the four nucleotides of DNA. Particular mutations, e.g., activated Ras mutations, can be unambiguously genotyped by mass spectrometry by measuring the differences in the mass of nucleic acids having alternative alleles, e.g. Ras alleles at the sites of activated Ras mutations. MALDI-TOF (Matrix Assisted Laser Desorption Ionization-Time of Flight) mass spectrometry technology is preferred for extremely precise determinations of molecular mass, such as Ras alleles, e.g., Ras alleles at the sites of activated Ras mutations. Numerous approaches to mutation analysis have been developed based on mass spectrometry. Preferred mass spectrometry-based methods of mutation detection include primer extension assays, which can also be utilized in combination with other approaches, such as traditional gel-based formats and microarrays.

Typically, the primer extension assay involves designing and annealing a primer to a template PCR amplicon upstream (5′) from a target mutation position, e.g. the activated Ras mutations. A mix of dideoxynucleotide triphosphates (ddNTPs) and/or deoxynucleotide triphosphates (dNTPs) are added to a reaction mixture containing template (e.g., a mutation-containing nucleic acid molecule which has typically been amplified, such as by PCR), primer, and DNA polymerase. Extension of the primer terminates at the first position in the template where a nucleotide complementary to one of the ddNTPs in the mix occurs. The primer can be either immediately adjacent (i.e., the nucleotide at the 3′ end of the primer hybridizes to the nucleotide next to the target mutation site) or two or more nucleotides removed from the mutation position. If the primer is several nucleotides removed from the target mutation position, the only limitation is that the template sequence between the 3′ end of the primer and the mutation position cannot contain a nucleotide of the same type as the one to be detected, or this will cause premature termination of the extension primer. Alternatively, if all four ddNTPs alone, with no dNTPs, are added to the reaction mixture, the primer will always be extended by only one nucleotide, corresponding to the target mutation position. In this instance, primers are designed to bind one nucleotide upstream from the mutation position (i.e., the nucleotide at the 3′ end of the primer hybridizes to the nucleotide that is immediately adjacent to the target mutation site on the 5′ side of the target mutation site). Extension by only one nucleotide is preferable, as it minimizes the overall mass of the extended primer, thereby increasing the resolution of mass differences between alternative mutation nucleotides. Furthermore, mass-tagged ddNTPs can be employed in the primer extension reactions in place of unmodified ddNTPs. This increases the mass difference between primers extended with these ddNTPs, thereby providing increased sensitivity and accuracy, and is particularly useful for typing heterozygous base positions. Mass-tagging also alleviates the need for intensive sample-preparation procedures and decreases the necessary resolving power of the mass spectrometer.

The extended primers can then be purified and analyzed by MALDI-TOF mass spectrometry to determine the identity of the nucleotide present at the target mutation position. In one method of analysis, the products from the primer extension reaction are combined with light absorbing crystals that form a matrix. The matrix is then hit with an energy source such as a laser to ionize and desorb the nucleic acid molecules into the gas-phase. The ionized molecules are then ejected into a flight tube and accelerated down the tube towards a detector. The time between the ionization event, such as a laser pulse, and collision of the molecule with the detector is the time of flight of that molecule. The time of flight is precisely correlated with the mass-to-charge ratio (m/z) of the ionized molecule. Ions with smaller m/z travel down the tube faster than ions with larger m/z and therefore the lighter ions reach the detector before the heavier ions. The time-of-flight is then converted into a corresponding, and highly precise, m/z. In this manner, mutations can be identified based on the slight differences in mass, and the corresponding time of flight differences, inherent in nucleic acid molecules having different nucleotides at a single base position. For further information regarding the use of primer extension assays in conjunction with MALDI-TOF mass spectrometry for mutational genotyping, see, e.g., Wise et al., “A standard protocol for single nucleotide primer extension in the human genome using matrix-assisted laser desorption/ionization time-of-flight mass spectrometry”, Rapid Commun Mass Spectrom. 2003; 17(11):1195-202.

Microfluidic devices (See e.g., U.S. Pat. Nos. 5,304,487, 5,110,745, 5,681,484, and 5,593,838) may be used in the detection of activated Ras mutations. Numerous methods are known in the art for determining the nucleotide occurrence for a particular mutation, e.g., activated Ras mutation, in a sample. Such methods can utilize one or more oligonucleotide probes or primers, including, for example, an amplification primer pair that selectively hybridizes to a target polynucleotide, which corresponds to one or more mutation positions. Oligonucleotide probes useful in practicing a method of the invention can include, for example, an oligonucleotide that is complementary to and spans a portion of the target polynucleotide, including the position of the mutation, wherein the presence of a specific nucleotide at the position is detected by the presence or absence of selective hybridization of the probe. Such a method can further include contacting the target polynucleotide and hybridized oligonucleotide with an endonuclease, and detecting the presence or absence of a cleavage product of the probe, depending on whether the nucleotide occurrence at the mutation site is complementary to the corresponding nucleotide of the probe. These oligonucleotides and probes are another embodiment of the present invention.

A microfluidic device useful for the methods of the present invention may allow for the application of an untreated biological sample on the device thus allowing isolation and purification of nucleic acids in one step. Alternatively, a microfluidic device for use in the methods of the present invention may combine isolation, purification and detection of nucleic acids, e.g., detection of activated Ras mutations, from application of an untreated biological sample to the device.

Mutations, e.g., activated Ras mutations, can also be scored by, direct DNA sequencing. A variety of automated sequencing procedures can be utilized ((1995) Biotechniques 19:448), including sequencing by mass spectrometry (see, e.g., PCT International Publication No. WO94/16101; Cohen et al., Adv. Chromatogr. 36:127-162 (1996); and Griffin et al., Appl. Biochem. Biotechnol. 38:147-159 (1993)). The nucleic acid sequences of the present invention enable one of ordinary skill in the art to readily design sequencing primers for such automated sequencing procedures. Commercial instrumentation, such as the Applied Biosystems 377, 3100, 3700, 3730, and 3730×1 DNA Analyzers (Foster City, Calif.), is commonly used in the art for automated sequencing.

Other approaches detect only that changes with respect to a reference sequence are present in a nucleic acid. Many of these methods are based on the formation of mismatches when both alleles, e.g., wild-type Ras and activated Ras, are present in the same sample. A currently very popular method is the use of denaturing high performance liquid chromatography to separate heteroduplex from homoduplex molecules (DHPLC; Oefner, P. J. et al. Am J Hum Genet 57 (Suppl.), A266 (1995)). Another method is denaturing gradient gel electrophoresis (DGGE) (Wartell et al., Nucleic Acids Res 18, 2699, (1990); Sheffield et al., Proc Natl Acad Sci USA 86, 232 (1989)). DGGE differentiates alleles based on the different sequence-dependent stabilities and melting properties inherent in polymorphic DNA and the corresponding differences in electrophoretic migration patterns in a denaturing gradient gel (Erlich, ed., PCR Technology, Principles and Applications for DNA Amplification, W.H. Freeman and Co, New York, 1992, Chapter 7). A variation is the clamped denaturing gel electrophoresis (CDGE; Sheffield et al., Am J Hum Genet 49, 699 (1991)), heteroduplex analysis (HA; White et al., Genomics 4, 560 (1992)) and chemical mismatch cleavage (CMC; Grompe et al. Proc Natl Acad Sci USA 86, 5888 (1989)). The use of proteins which recognize nucleotide mismatches, such as the E. coli mutS protein may help in detecting mismatched DNA molecules (Modrich, Ann. Rev. Genetics, 25, 229 (1991)). In the mutS assay, the protein binds only to sequences that contain a nucleotide mismatch in a heteroduplex between mutant and wild-type sequences. RNase protection assays are another option (Finkelstein et al., Genomics 7, 167 (1990)). The RNAse protection assay involves cleavage of the mutant fragment into two or more smaller fragments. Another way is to make use of the single-stranded conformation polymorphism assay (SSCP; Orita et al., Proc Natl Acad Sci USA 86, 2766 (1989)). Variations in the DNA sequence of the ras gene from the reference sequence will be detected due to a shifted mobility of the corresponding DNA-fragments in SSCP gels. SSCP detects bands which migrate differently because the variation causes a difference in single strand, intra-molecular base pairing.

Sequence-specific ribozymes (e.g., U.S. Pat. No. 5,498,531) can also be used to score mutations based on the development or loss of a ribozyme cleavage site. Perfectly matched sequences can be distinguished from mismatched sequences by nuclease cleavage digestion assays or by differences in melting temperature. If the mutation, e.g., in the ras genome which leads to an activated Ras protein, affects a restriction enzyme cleavage site, the mutation can be identified by alterations in restriction enzyme digestion patterns, and the corresponding changes in nucleic acid fragment lengths determined by gel electrophoresis.

Mutations of interest including activating ras mutations, can be simultaneously detected in a high throughput assay conducted using microbeads labeled with different spectral property and/or fluorescent (or colorimetric) intensity. For example, polystyrene microspheres are provided by Luminex Corp, Austin, Tex. that are internally dyed with two spectrally distinct fluorochromes. Using precise ratios of these fluorochromes, a large number of different fluorescent bead sets (e.g., 100 sets) can be produced. Each set of the beads can be distinguished by its spectral address, a combination of which allows for measurement of a large number of analytes in a single reaction vessel. A third fluorochrome coupled to a reporter molecule quantifies the biomolecular interaction that has occurred at the microsphere surface. These different fluorescent bead sets can be used to label the reference nucleic acids targeting different mutation sites on a test DNA by using standard nucleic acid chemical synthesis methods or by PCR amplification using primers labeled with the beads (e.g., primers modified with 5′ amine for coupling to carboxylated microsphere or bead).

Further PCR-based techniques for identification of genetic mutations present in a cell include, for example, differential display (Liang and Pardee, Science 257:967-971 (1992)); amplified fragment length polymorphism (iAFLP) (Kawamoto et al., Genome Res. 12:1305-1312 (1999)); BeadArray™. technology (Illumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression (BADGE), using the commercially available Luminex 100 LabMAP system and multiple color-coded microspheres (Luminex Corp., Austin, Tex.) in a rapid assay for gene expression (Yang et al., Genome Res. 11:1888-1898 (2001)); and high coverage expression profiling (HiCEP) analysis (Fukumura et al., Nucl. Acids. Res. 31(16) e94 (2003)).

Other suitable amplification methods include, but are not limited to ligase chain reaction (LCR) (see Wu and Wallace (1989) Genomics 4:560, Landegren et al. (1988) Science 241:1077, and Barringer et al. (1990) Gene 89:117), transcription amplification (Kwoh et al. (1989) Proc. Natl. Acad. Sci. USA 86:1173), self-sustained sequence replication (Guatelli et al. (1990) Proc. Nat. Acad. Sci. USA 87:1874), dot PCR, and linker adapter PCR, etc.

Detection of mutations, e.g., activated Ras mutations, can include one or more of the steps of collecting a biological sample from a human subject (e.g., sample of tissues, cells, fluids, secretions, etc.), isolating nucleic acids (e.g., genomic DNA, mRNA or both) from the cells of the sample, contacting the nucleic acids with one or more primers which specifically hybridize to a region of the isolated nucleic acid containing a target mutations under conditions such that hybridization and amplification of the target nucleic acid region occurs, and determining the nucleotide present at the mutational position of interest, or, in some assays, detecting the presence or absence of an amplification product (assays can be designed so that hybridization and/or amplification will only occur if a particular allele is present or absent at the site of mutation, e.g., activated Ras mutation). In some assays, the size of the amplification product is detected and compared to the length of a control sample; for example, deletions and insertions can be detected by a change in size of the amplified product compared to a normal genotype.

Laser Capture Microdissection can be used to specifically examine tumor cells in a biopsy, and examine the nucleic acid and/or protein content of those tumor cells (Emmert-Buck, et al., Science 274:998-1001, 1996; Bonner, et al., Science 278:1481-3, 1997). The expression levels of specific ras mRNA and/or Ras proteins (e.g. activated, wild-type) can be quantitatively examined. These levels can further be compared to non-tumor cells within the same biopsy sample to identify increased expression in a tumor cell. Alternatively, or in addition, specific downstream effectors known to be induced by Ras, or PI₃K can be examined and compared in the tumor versus non-tumor cells of a biopsy, to determine if a Ras and/or PI₃K signal transduction pathway is activated in the tumor cells.

### Protein Based Methods

Protein expression, e.g. activated Ras expression, can be detected and quantified, where applicable, using various well-known immunological assays. Immunological assays refer to assays that utilize an antibody (e.g., polyclonal, monoclonal, chimeric, humanized, scFv, and fragments thereof) that specifically binds to activated Ras proteins over wild-type Ras proteins. Such antibodies are herein referred to as activated Ras antibodies. A number of well-established immunological assays suitable for the practice of the present invention are known, and include ELISA, radioimmunoassay (RIA), immunoprecipitation, immunofluorescence, and Western blotting.

The activated Ras antibodies (preferably anti-mammalian; more preferably anti-human), polyclonal or monoclonal, to be used in the immunological assays utilized in the methods of the present invention are commercially available from a variety of commercial suppliers, e.g., AbCam (Cambridge UK and Cambridge, Mass.), Upstate Group (Charlottesville, Va.), and Pierce Biotechnology, Inc. (Rockford, Ill.). U.S. Pat. Publ. No. 20050288492 discloses antibodies to activated Ras. Active Motif (Carlsbad, Calif.) makes available ELISA kits for detection of activated Ras. Alternatively, antibodies may be produced by methods well known to those skilled in the art, e.g., as described in Harlow et al., Antibodies: A Laboratory Manual, 2nd Ed; Cold Spring Harbor Laboratory Press, Cold Spring Harbor, N.Y. (1988). For example, monoclonal antibodies to activated Ras, preferably mammalian; more preferably human, can be produced by generation of hybridomas in accordance with known methods. Hybridomas formed in this manner are then screened using standard methods, such as ELISA, to identify one or more hybridomas that produce an antibody that specifically binds to the antigen of interest. Full-length antigen of interest, e.g. activated Ras, may be used as the immunogen, or, alternatively, antigenic peptide fragments of the antigen of interest may be used.

As an alternative to preparing monoclonal antibody-secreting hybridomas, a monoclonal antibody to the antigen of interest, e.g. activated Ras, may be identified and isolated by screening a recombinant combinatorial immunoglobulin library (e.g., an antibody phage display library) to thereby isolate immunoglobulin library members that bind to the antigen of interest, e.g. activated Ras. Kits for generating and screening phage display libraries are commercially available from, e.g., Dyax Corp. (Cambridge, Mass.) and Maxim Biotech (South San Francisco, Calif.). Additionally, examples of methods and reagents particularly amenable for use in generating and screening antibody display libraries can be found in the literature.

Polyclonal sera and antibodies may be produced by immunizing a suitable subject, such as a rabbit, with the antigen of choice, e.g. activated Ras, preferably mammalian; more preferably human, or an antigenic fragment thereof. The antibody titer in the immunized subject may be monitored over time by standard techniques, such as with ELISA, using immobilized marker protein. If desired, the antibody molecules directed against the antigen of interest, e.g. activated Ras, may be isolated from the subject or culture media and further purified by well-known techniques, such as protein A chromatography, to obtain an IgG fraction.

Fragments of antibodies to the antigen of interest, e.g. activated Ras, may be produced by cleavage of the antibodies in accordance with methods well known in the art. For example, immunologically active F(ab′) and F(ab′)₂ fragments may be generated by treating the antibodies with an enzyme such as pepsin. Additionally, chimeric, humanized, and single-chain antibodies to the antigen of interest, comprising both human and nonhuman portions, may be produced using standard recombinant DNA techniques. Humanized antibodies to the antigen of interest may also be produced using transgenic mice that are incapable of expressing endogenous immunoglobulin heavy and light chain genes, but which can express human heavy and light chain genes.

Antibody production is useful to utilizing some of the methods of the present invention. Antibodies can be prepared against the immunogen (e.g. activated Ras protein), or any portion thereof, for example a synthetic peptide based on the sequence. As stated above, antibodies are used in assays and are therefore used in determining if the appropriate enzyme has been isolated. Antibodies can also be used for removing enzymes from red cell suspensions after enzymatic conversion. Immunogens can be used to produce antibodies by standard antibody production technology well known to those skilled in the art as described generally in Harlow and Lane, Antibodies: A Laboratory Manual, Cold Springs Harbor Laboratory, Cold Spring Harbor, N.Y., 1988 and Borrebaeck, Antibody Engineering—A Practical Guide, W. H. Freeman and Co., 1992. Antibody fragments can also be prepared from the antibodies and include Fab, F(ab′)₂, and Fv by methods known to those skilled in the art.

In the immunological assays utilized in the methods of the present invention, the antigen, e.g. activated Ras, is typically detected directly (i.e., the antibody to the antigen of interest is labeled) or indirectly (i.e., a secondary antibody that recognizes the antibody to the antigen of interest is labeled) using a detectable label. The particular label or detectable group used in the assay is usually not critical, as long as it does not significantly interfere with the specific binding of the antibodies used in the assay.

The immunological assays utilized in the methods of the present invention may be competitive or noncompetitive. In competitive assays, the amount of a specific Ras protein (e.g. an activated Ras protein) in a sample is measured indirectly by measuring the amount of added (exogenous) specific Ras protein displaced from a capture agent, i.e. an anti-activated Ras antibody, by the specific Ras protein in the sample. In noncompetitive assays, the amount of a specific Ras protein in a sample is directly measured. In a preferred noncompetitive “sandwich” assay, the capture agent (e.g., a first antibody) is bound directly to a solid support (e.g., membrane, microtiter plate, test tube, dipstick, glass or plastic bead) where it is immobilized. The immobilized agent then captures any antigen of interest present in the sample. The immobilized antigen of interest can then be detected using a second labeled antibody to the antigen of interest. Alternatively, the second antibody can be detected using a labeled secondary antibody that recognizes the second antibody.

A preferred method of measuring the expression of the antigen of interest, e.g. a Ras protein, is by antibody staining with an antibody that binds specifically to the antigen employing a labeling strategy that makes use of luminescence or fluorescence. Such staining may be carried out on fixed tissue or cells that are ultimately viewed and analyzed under a microscope. Staining carried out in this manner can be scored visually or by using optical density measurements. Staining may also be carried out using either live or fixed whole cells in solution, e.g. cells isolated from blood or tumor biopsy. Such cells can be analyzed using a fluorescence activated cell sorter (FACS), which can determine both the number of cells stained and the intensity of the luminescence or fluorescence. Such techniques are well known in the art, and exemplary techniques are described in Luwor et al. ((2001), Cancer Res. 61:5355-61). One of skill in the art will realize that other techniques of detecting expression of a specific protein might be more or less sensitive than these techniques. As described herein, cells express little or no antigen if little or no antigen can be detected using an antibody staining technique that relies on luminescence or fluorescence.

Alternatively, expression of specific Ras proteins (e.g. activated Ras) in cells, particularly tumor cells, can be detected in vivo in a subject by introducing into the subject a labeled antibody to the specific Ras protein (e.g. activated Ras protein). For example, the antibody can be labeled with a radioactive marker whose presence and location in a subject can be detected by standard imaging techniques.

In one preferred embodiment, immunohistochemistry (“IHC”) and immunocytochemistry (“ICC”) techniques, for example, may be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of a specific antibody, wherein antibodies are used to specifically target molecules inside or on the surface of cells. The antibody typically contains a marker that will undergo a biochemical reaction, and thereby experience a change color, upon encountering the targeted molecules. In some instances, signal amplification may be integrated into the particular protocol, wherein a secondary antibody, that includes the marker stain, follows the application of a primary specific antibody.

Immunohistochemical assays are known to those of skill in the art (e.g., see Jalkanen, et al., J. Cell. Biol. 101:976-985 (1985); Jalkanen, et al., J. Cell. Biol. 105:3087-3096 (1987).

Typically, for immunohistochemistry, tissue sections are obtained from a patient and fixed by a suitable fixing agent such as alcohol, acetone, and paraformaldehyde, to which is reacted an antibody. Conventional methods for immunohistochemistry are described in Harlow and Lane (eds) (1988) In “Antibodies A Laboratory Manual”, Cold Spring Harbor Press, Cold Spring Harbor, N.Y.; Ausbel et al (eds) (1987), in Current Protocols In Molecular Biology, John Wiley and Sons (New York, N.Y.). Biological samples appropriate for such detection assays include, but are not limited to, cells, tissue biopsy, whole blood, plasma, serum, sputum, cerebrospinal fluid, breast aspirates, pleural fluid, urine and the like. In one embodiment, the biological sample contains tumor cells. In another embodiment, the biological sample contains pre-tumor cells.

For direct labeling techniques, a labeled antibody is utilized. For indirect labeling techniques, the sample is further reacted with a labeled substance.

Alternatively, immunocytochemistry may be utilized. In general, cells are obtained from a patient and fixed by a suitable fixing agent such as alcohol, acetone, and paraformaldehyde, to which is reacted an antibody. Methods of immunocytological staining of human samples is known to those of skill in the art and described, for example, in Brauer et al., 2001 (FASEB J, 15, 2689-2701), Smith-Swintosky et al., 1997.

Immunological methods of the present invention are advantageous because they require only small quantities of biological material. Such methods may be done at the cellular level and thereby necessitate a minimum of one cell. Preferably, several cells are obtained from a patient affected with or at risk for developing cancer and assayed according to the methods of the present invention.

### Mutation Detection Kits and Systems

Accordingly, the present invention further relates to kits and systems for detection of mutations or abnormalities in a cell which lead to or indicate the apoptosis sensitivity to PKC-delta inhibition described herein. By way of non-limiting example, mutation detection, e.g., activated Ras mutation detection, kits and systems, Ras expression detection and quantitation kits, kits for Ras effector activity analysis. These kits and systems include but are not limited to, packaged probe and primer sets (e.g., TaqMan probe/primer sets), arrays/microarrays of nucleic acid molecules, and beads that contain one or more probes, primers, or other detection reagents for detecting activated Ras mutations, and specific kinase assay and/or downstream effector detection. The kits/systems can optionally include various electronic hardware components; for example, arrays (“DNA chips”) and microfluidic systems (“lab-on-a-chip” systems, biomedical micro-electro-mechanical systems (bioMEMs), or multicomponent integrated systems) provided by various manufacturers typically comprise hardware components. Other kits/systems (e.g., probe/primer sets, etc.) may not include electronic hardware components, but may be comprised of, for example, one or more mutation detection reagents (along with, optionally, other biochemical reagents) packaged in one or more containers, and containing all relevant and appropriate content and instruction materials.

### Method of Treatment

The present invention provides methods to treat subjects with cancer or a non-malignant proliferative disorder, wherein the abnormally proliferating cells have activated Ras mutations, and/or over-express wild-type Ras, and/or have over-active Ras, and/or otherwise have constitutive activation of effector pathways downstream of Ras (e.g. the PI₃K signaling pathway) with a PKC-delta inhibitor.

The treatment comprises administration of one or more PKC-delta inhibitor(s) to the subject by a method which results in inhibition of the PKC-delta in the cell or cells which have aberrantly increased Ras signaling. In one embodiment, administration is by a method which results in contact of the inhibitor with the cells which have aberrantly increased Ras signaling. The inhibitor(s) may function to inhibit at one or more levels in the cell (e.g. PKC-delta gene expression, protein translation, or protein function (kinase activity).

In a preferred embodiment, the PKC-delta inhibitor is selective for PKC-delta. In another embodiment, the PKC-delta inhibitor inhibits PKC-delta and one or more other PKC isoforms. The inhibitor may be a small molecule, a peptide antagonist, a competitive inhibitor, an irreversible inhibitor, a nucleic acid, including siRNA or antisense, an antibody, an antibody fragment, or an aptamer. In one embodiment, the subject is administered PKC-delta inhibitors in combination with other treatments for cancer, including chemotherapeutics, immunotherapeutics, anti-angiogenesis therapeutics and targeted therapeutics. Targeted therapeutics may include therapeutics targeted to other genes and gene products inappropriately expressed or mutated in cancer. Examples of targeted therapeutics include, but are not limited to, ErbB targeting therapeutics, e.g., EGFR and/or HER2 targeting agents.

In one embodiment, administration of the PKC-delta inhibitor results in a decrease in PKC-delta activity of at least 10% compared to an appropriate control, e.g., comparable PKC-delta activity in a subject who has not been administered a PKC-delta inhibitor. Preferably, PKC-delta activity is decreased at least about 20, about 30, about 40, about 50, about 60, about 70, about 80, about 90, about 100, about 150, or about 200% or more, compared to a control.

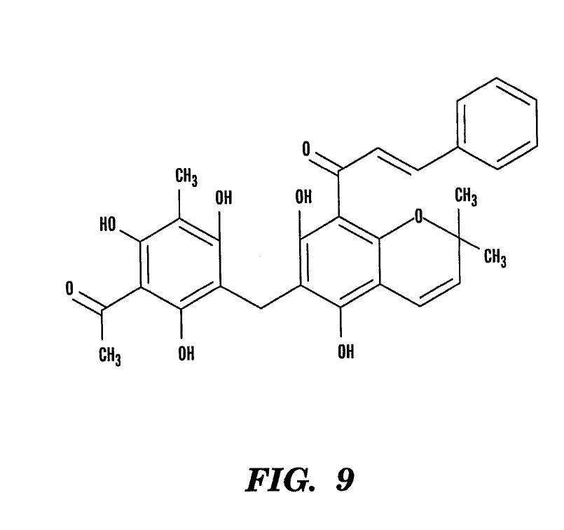

In a preferred embodiment, the PKC-delta inhibitor the selective PKC-delta inhibitor KAI-9803 (KAI Pharmaceuticals, Inc., South San Francisco, Calif.). Another PKC-delta selective inhibitor is Rottlerin (mallatoxin) and functional derivatives thereof. The structure of Rottlerin is shown in FIG. 9. One can make derivatives based upon the structure of mallatoxin or balanol wherein the core structure is substitutes by C₁-C₆ groups such as aslkyl, aryl, alkenyl, alkoxy, heteroatoms such as S, N, O, and halogens.

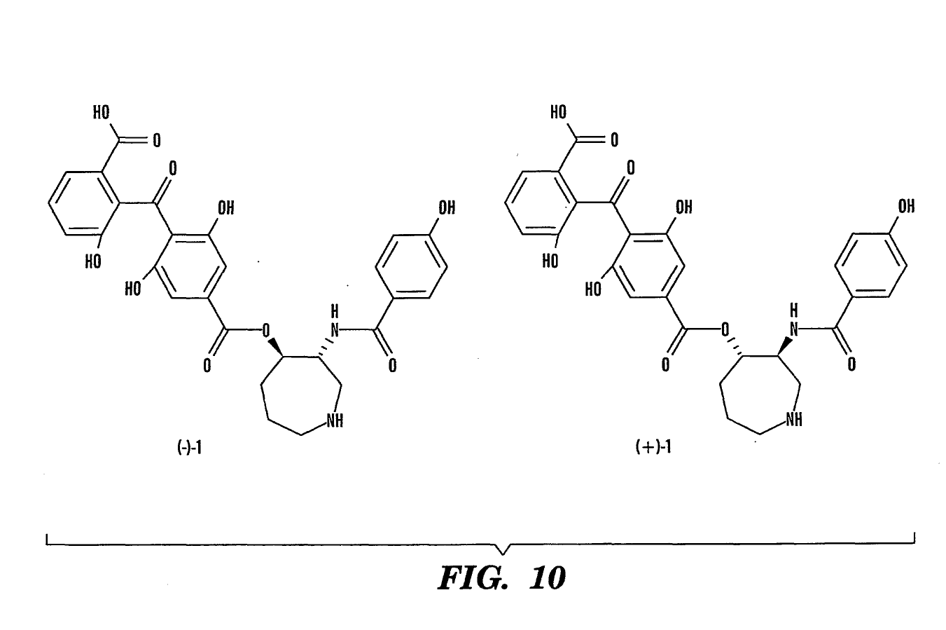

Another PKC-delta selective inhibitor is Balanol and Balanol analogs (perhydroazepine-substitution analogs). The chemical structure of balanol is shown in FIG. 10. Balanol and perhydroazepine-substitution analogs are known to the skilled practitioner. Several moieties and inhibitory activities of Balanol and various substitutions analogs are described in Table 2 below.

TABLE 2

| | | | IC₅₀ | |
|---|---|---|---|---|
| Analog | δ > α | δ > PKA | (PKCδ) | Moiety |
| (−) 14 | 75x | 5x | 0.09 | 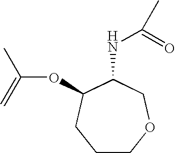 |
| (−) 23 | 102x | 7x | 0.05 | 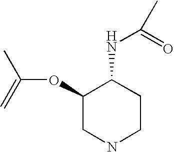 |
| Balanol | 9x | 12x | 0.004 | 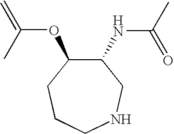 |
| (−) 24 | 40x | 30x | 0.001 | 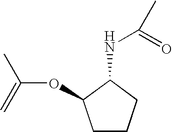 |

δ > α:: Ratio of IC₅₀ for PKCδ to IC₅₀ PKCα
δ > PKA: Ratio of IC₅₀ for PKCδ to IC₅₀ PKA
Balanol: (−) enantiomer is the one with the inhibitory activity
Balanol perhydroazepine-substitution Analogs 14, 23, 24 (Lai Y S, Mendoza J S, Jagdmann G E, Jr., Menaldino D S et al. J Med. Chem. 40:226-35 (1997)).

Int'l Pat. Appl. WO04/078118 and U.S. Pat. Nos. 6,828,327, 6,723,830, 6,686,373 and 5,843,935 provide PKC inhibitors, including PKC inhibitors selective for inhibition of PKC-delta. Other PKC inhibitors may be useful in the methods of the present invention, including, phytosphingosine, phytospinogsine-1-phosphate, sphinganine, sphinganine-1, lysosphingoids, sphingosine, sphingosine-1-phosphate, curcumin and curcuminoids, tetrahydrocurcumin, apigenin, safingol, an optical isomer (the L-threo enantiomer) of dihydrosphingosine and W-7. Phytosphingosine is the preferred ceramide for this invention. However, other ceramides selected from the group N-tetracosanoyl phytosphingosine, N-stearoyl phytosphingosine, N-oleoyl phytsosphingosine, N-linoleoyl-phytosphingosine, N-(2-hydroxytetracosanoy-1), phytosphingosine, N-(2-hydroxyoctdecanoyl) phytosphingosine, N-phytosphingosine, 22(2hydroxyoctdecanoyl) hydroxyoctdecanoyl) phytosphingosine, N-(27-stearoyloxy-hepatoaconsanoyl) phtosphingosine, N-(27-oleoyloxheptacosanoyl) phytosphingosine, N (27-linoleoyoxyheptaconsa-noyl) phytosphingosine, N-(23-stearoyloxytricosanoyl) phytosphingosine may be used. Staurosporine derivatives may be useful, including UCN-01 (7-OH-staurosporine) and those disclosed in U.S. Pat. No. 5,093,330. Such compounds can be administered, e.g., in the form as disclosed in WO99/48896.

Other PKC inhibitors include PKC-412 (N -benzoylstaurosporine), bryostatin 1 (Macrocyclic lactone), perifosine, limofosine, Ro 31-8220, Ro 32-0432, GO 6976, ISIS-3521 (CGP 64128A), the macrocyclic bis (indolyl) maleimides (LY-333531, LY-379196, LY-317615; Eli Lilly and Co), BAY 43-9006 (Sorafenib, Onyx Pharmaceuticals), RO32-0432 (Bisindolylmaleimide tertiary amine), RO318220, Flavopiridol (L86-8275)₁, CGP52421, GO 6976. Midostaurin is a derivative of the naturally occurring alkaloid staurosporine with the chemical name (N-[(9S,10R,11R,13R)-2,3,10,11,12,13-hexahydro-10-methoxy-9-methyl-1-oxo-9,13-epoxy-1H,9H-diindolo[1,2,3-gh:3′,2′,1′-1m]pyrrolo[3,4-j][1,7]benzodia-zonin-11-yl]-N-methylbenzamide), and has been specifically described in the European patent No. 0 296 110, U.S. Pat. No.5,093,330 and Japanese Patent No. 2 708 047. Midostaurin was originally identified as an inhibitor of protein kinase C (PKC) (Meyer T, Regenass U, Fabbro D, et al: Int J Cancer 43: 851-856, 1989).

In another embodiment, compounds useful in the method of the present invention are antibodies which interfere with kinase signaling via PKC-delta, including monoclonal, chimeric, humanized, recombinant antibodies and fragment thereof which are characterized by their ability to inhibit the kinase activity of PKC-delta and which have low toxicity. One can also use nucleic acid based approaches to inhibit expression or protein production of PKC-delta. Such methods include, without limitation, interfering RNA, missense, and antisense techniques. An example of use of siRNA to inhibit PKC-delta is provided in the Examples section herein.

Neutralizing antibodies are readily raised in animals such as rabbits or mice by immunization with PKC-delta. Immunized mice are particularly useful for providing sources of B cells for the manufacture of hybridomas, which in turn are cultured to produce large quantities of anti-PKC-delta monoclonal antibodies. Chimeric antibodies are immunoglobin molecules characterized by two or more segments or portions derived from different animal species. Generally, the variable region of the chimeric antibody is derived from a non-human mammalian antibody, such as murine monoclonal antibody, and the immunoglobin constant region is derived from a human immunoglobin molecule. Preferably, both regions and the combination have low immunogenicity as routinely determined. Humanized antibodies are immunoglobin molecules created by genetic engineering techniques in which the murine constant regions are replaced with human counterparts while retaining the murine antigen binding regions. The resulting mouse-human chimeric antibody should have reduced immunogenicity and improved pharmacokinetics in humans. Preferred examples of high affinity monoclonal antibodies and chimeric derivatives thereof, useful in the methods of the present invention, are described in the European Patent Application EP 186,833; PCT Patent Application WO 92/16553; and U.S. Pat. No. 6,090,923.

In connection with the administration of the drug, an “effective amount” indicates an amount that results in a beneficial effect for at least a statistically significant fraction of patients, such as a improvement of symptoms, a cure, a reduction in disease load, reduction in tumor mass or cell numbers, extension of life, improvement in quality of life, or other effect generally recognized as positive by medical doctors familiar with treating the particular type of disease or condition.

The effective dosage of active ingredient employed may vary depending on the particular compound employed, the mode of administration and the severity of the condition being treated. The skilled artisan is aware of the effective dose for each patient, which may vary with disease severity, individual genetic variation, or metabolic rate. However, in general, satisfactory results are obtained when the compounds of the invention are administered at a daily dosage of from about 0.5 to about 1000 mg/kg of body weight, optionally given in divided doses two to four times a day, or in sustained release form. The total daily dosage is projected to be from about 1 to 1000 mg, preferably from about 2 to 500 mg. Dosage forms suitable for internal use comprise from about 0.5 to 1000 mg of the active compound in intimate admixture with a solid or liquid pharmaceutically acceptable carrier. This dosage regimen may be adjusted to provide the optimal therapeutic response. For example, several divided doses may be administered daily or the dose may be proportionally reduced as indicated by the exigencies of the therapeutic situation.

The pharmaceutical composition in which the PKC-delta inhibitor is administered, and the route of administration will be determined by the skilled practitioner, who will take into consideration variables such as tumor type and location, and disease stage. Possible routes of administration include, without limitation, intravenous (I.V.), intramuscular (I.M.), subcutaneous (S.C.), intradermal (I.D.), intraperitoneal (I.P.), intrathecal (I.T.), intrapleural, intrauterine, rectal, vaginal, topical, intratumor and the like. The compounds of the invention can be administered parenterally by injection or by gradual infusion over time and can be delivered by peristaltic means.

Administration may be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays, for example, or using suppositories. For oral administration, the compounds of the invention are formulated into conventional oral administration forms such as capsules, tablets and tonics.

For topical administration, the pharmaceutical composition (inhibitor of kinase activity) is formulated into ointments, salves, gels, or creams, as is generally known in the art.

The therapeutic compositions of this invention, e.g., PKC-delta inhibitors, are conventionally administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluents; i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. Precise amounts of active ingredient required to be administered depend on the judgment of the practitioner and are peculiar to each individual.

The therapeutic composition useful for practicing the methods of the present invention, e.g. PKC-delta inhibitors, are described herein. Any formulation or drug delivery system containing the active ingredients, which is suitable for the intended use, as are generally known to those of skill in the art, can be used. Suitable pharmaceutically acceptable carriers for oral, rectal, topical or parenteral (including inhaled, subcutaneous, intraperitoneal, intramuscular and intravenous) administration are known to those of skill in the art. The carrier must be pharmaceutically acceptable in the sense of being compatible with the other ingredients of the formulation and not deleterious to the recipient thereof.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects.

Formulations suitable for parenteral administration conveniently include sterile aqueous preparation of the active compound which is preferably isotonic with the blood of the recipient. Thus, such formulations may conveniently contain distilled water, 5% dextrose in distilled water or saline. Useful formulations also include concentrated solutions or solids containing the compound which upon dilution with an appropriate solvent give a solution suitable for parental administration above.

For enteral administration, a compound can be incorporated into an inert carrier in discrete units such as capsules, cachets, tablets or lozenges, each containing a predetermined amount of the active compound; as a powder or granules; or a suspension or solution in an aqueous liquid or non-aqueous liquid, e.g., a syrup, an elixir, an emulsion or a draught. Suitable carriers may be starches or sugars and include lubricants, flavorings, binders, and other materials of the same nature.

A tablet may be made by compression or molding, optionally with one or more accessory ingredients. Compressed tablets may be prepared by compressing in a suitable machine the active compound in a free-flowing form, e.g., a powder or granules, optionally mixed with accessory ingredients, e.g., binders, lubricants, inert diluents, surface active or dispersing agents. Molded tablets may be made by molding in a suitable machine, a mixture of the powdered active compound with any suitable carrier.

A syrup or suspension may be made by adding the active compound to a concentrated, aqueous solution of a sugar, e.g., sucrose, to which may also be added any accessory ingredients. Such accessory ingredients may include flavoring, an agent to retard crystallization of the sugar or an agent to increase the solubility of any other ingredient, e.g., as a polyhydric alcohol, for example, glycerol or sorbitol.

Formulations for rectal administration may be presented as a suppository with a conventional carrier, e.g., cocoa butter or Witepsol S55 (trademark of Dynamite Nobel Chemical, Germany), for a suppository base.

Formulations for oral administration may be presented with an enhancer. Orally-acceptable absorption enhancers include surfactants such as sodium lauryl sulfate, palmitoyl carnitine, Laureth-9, phosphatidylcholine, cyclodextrin and derivatives thereof; bile salts such as sodium deoxycholate, sodium taurocholate, sodium glycochlate, and sodium fusidate; chelating agents including EDTA, citric acid and salicylates; and fatty acids (e.g., oleic acid, lauric acid, acylcarnitines, mono- and diglycerides). Other oral absorption enhancers include benzalkonium chloride, benzethonium chloride, CHAPS (3-(3-cholamidopropyl)-dimethylammonio-1-propanesulfonate), Big-CHAPS (N,N-bis(3-D-gluconamidopropyl)-cholamide), chlorobutanol, octoxynol-9, benzyl alcohol, phenols, cresols, and alkyl alcohols. An especially preferred oral absorption enhancer for the present invention is sodium lauryl sulfate.

Alternatively, the compound may be administered in liposomes or microspheres (or microparticles). Methods for preparing liposomes and microspheres for administration to a patient are well known to those of skill in the art. U.S. Pat. No. 4,789,734, the contents of which are hereby incorporated by reference, describes methods for encapsulating biological materials in liposomes. Essentially, the material is dissolved in an aqueous solution, the appropriate phospholipids and lipids added, along with surfactants if required, and the material dialyzed or sonicated, as necessary. A review of known methods is provided by G. Gregoriadis, Chapter 14, “Liposomes,” Drug Carriers in Biology and Medicine, pp. 287-341 (Academic Press, 1979).

Microspheres formed of polymers or proteins are well known to those skilled in the art, and can be tailored for passage through the gastrointestinal tract directly into the blood stream. Alternatively, the compound can be incorporated and the microspheres, or composite of microspheres, implanted for slow release over a period of time ranging from days to months. See, for example, U.S. Pat. Nos. 4,906,474, 4,925,673 and 3,625,214, and Jein, TIPS 19:155-157 (1998), the contents of which are hereby incorporated by reference.

In one embodiment, the tyrosine kinase inhibitor of the present invention can be formulated into a liposome or microparticle which is suitably sized to lodge in capillary beds following intravenous administration. When the liposome or microparticle is lodged in the capillary beds surrounding ischemic tissue, the agents can be administered locally to the site at which they can be most effective. Suitable liposomes for targeting ischemic tissue are generally less than about 200 nanometers and are also typically unilamellar vesicles, as disclosed, for example, in U.S. Pat. No. 5,593,688 to Baldeschweiler, entitled “Liposomal targeting of ischemic tissue,” the contents of which are hereby incorporated by reference.

Preferred microparticles are those prepared from biodegradable polymers, such as polyglycolide, polylactide and copolymers thereof. Those of skill in the art can readily determine an appropriate carrier system depending on various factors, including the desired rate of drug release and the desired dosage.

In one embodiment, the formulations are administered via catheter directly to the inside of blood vessels. The administration can occur, for example, through holes in the catheter. In those embodiments wherein the active compounds have a relatively long half life (on the order of 1 day to a week or more), the formulations can be included in biodegradable polymeric hydrogels, such as those disclosed in U.S. Pat. No. 5,410,016 to Hubbell et al. These polymeric hydrogels can be delivered to the inside of a tissue lumen and the active compounds released over time as the polymer degrades. If desirable, the polymeric hydrogels can have microparticles or liposomes which include the active compound dispersed therein, providing another mechanism for the controlled release of the active compounds.

The formulations may conveniently be presented in unit dosage form and may be prepared by any of the methods well known in the art of pharmacy. All methods include the step of bringing the active compound into association with a carrier which constitutes one or more accessory ingredients. In general, the formulations are prepared by uniformly and intimately bringing the active compound into association with a liquid carrier or a finely divided solid carrier and then, if necessary, shaping the product into desired unit dosage form.

The formulations may further include one or more optional accessory ingredient(s) utilized in the art of pharmaceutical formulations, e.g., diluents, buffers, flavoring agents, binders, surface active agents, thickeners, lubricants, suspending agents, preservatives (including antioxidants) and the like.

Compounds of the present methods, e.g., PKC inhibitors, e.g., PKC-delta inhibitors, may be presented for administration to the respiratory tract as a snuff or an aerosol or solution for a nebulizer, or as a microfine powder for insufflation, alone or in combination with an inert carrier such as lactose. In such a case the particles of active compound suitably have diameters of less than 50 microns, preferably less than 10 microns, more preferably between 2 and 5 microns.

Generally for nasal administration a mildly acid pH will be preferred. Preferably the compositions of the invention have a pH of from about 3 to 5, more preferably from about 3.5 to about 3.9 and most preferably 3.7. Adjustment of the pH is achieved by addition of an appropriate acid, such as hydrochloric acid.

The preparation of a pharmaceutical composition that contains active ingredients dissolved or dispersed therein is well understood in the art and need not be limited based on formulation. Typically such compositions are prepared as injectables either as liquid solutions or suspensions, however, solid forms suitable for solution, or suspensions, in liquid prior to use can also be prepared. The preparation can also be emulsified.

The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

The PKC-delta inhibitors for use in the methods of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions can also contain liquid phases in addition to and to the exclusion of water. Exemplary of such additional liquid phases are glycerin, vegetable oils such as cottonseed oil, and water-oil emulsions.

### Definitions

The term “aberrantly increased Ras signaling” as used herein refers to a statistically significant increase in Ras signaling in one or more cells (e.g. tumor or pre-tumor cells) as measured by a determination of the percentage of Ras in the activated state and/or activity or one or more downstream effectors of Ras. Such determination is further made by comparison of similar measurements made in a similar cells type under appropriate conditions. Increased activity may be surmised by detection of a known activated Ras mutant, at the nucleic acid or the protein level. Increased activity of Ras may also be surmised by a detected abnormal increase in the activity and/or presence of a known activator of Ras or abnormal decrease in known deactivator of Ras, as described herein. Verification of actual increase in Ras signaling may be used to confirm such surmisal.

The term “PKC delta inhibitor” as used herein refers to a molecule having the ability to inhibit a biological function of a native PKC delta, including mutant PKC delta. Accordingly, the term “inhibitor” is defined in the context of the biological role of PKC delta. While preferred inhibitors herein specifically interact with, e.g. bind to, a PKC delta, molecules that inhibit PKC delta biological activity by interacting with other members of the PKC delta signal transduction pathway are also specifically included within this definition. Useful PKC delta inhibitors may selectively inhibit PKC delta, may selectively inhibit calcium-independent or novel PKC isoforms or may broadly inhibit PKC isoforms. A preferred PKC delta biological activity inhibited by a PKC delta inhibitor is associated with the development, growth, or spread of a tumor or associated with the development or proliferation. PKC delta inhibitors, without limitation, include peptides, non-peptide small molecules, antibodies, antibody fragments, antisense molecules, and oligonucleotide decoys. Some PKC delta inhibitors may function by more than one mechanism to inhibit overall PKC delta activity in a cell.

A “PKC delta targeting treatment” is the use of one or more PKC delta inhibitors to therapeutically reduce PKC delta activity in a cell. The PKC delta inhibitors may preferably be agents that selectively inhibit PKC-delta. As used herein, an agent that “selectively inhibits” PKC-delta means an agent that reduces the activity of PKC-delta more than it reduces the activity of any other PKC isoform.

As used herein, the term “subject” or “patient” refers to any mammal. The subject is preferably human, but can also be a mammal in need of veterinary treatment, e.g. domestic animals, farm animals, and laboratory animals. For example, the subject may be a subject diagnosed with a benign or malignant tumor, a cancer or a hyperplasia. The subject may be a cancer patient who is receiving treatment modalities against cancer or has undergone a regimen of treatment, e.g., chemotherapy, radiation and/or surgery. The subject may be a cancer patient whose cancer appears to be regressing.

As used herein, the phrase “expression” is used to refer to the transcription of a gene product into mRNA (gene expression) and is also used to refer to the expression of the protein encoded by the gene.

As used herein the term “over-expression” is used to refer to increased production of a specific mRNA and/or protein in a cell, wherein the actual mRNA and protein product do not contain activating mutations. As used herein, the term “over-activation”, as used to refer to Ras or an upstream or downstream effector, is used to refer to increased signaling through an otherwise non-activated form of a pathway member. Over-activation of a molecule typically results from increased activation (e.g. upstream signaling) or decreased de-activation (e.g. downstream negative regulation) of the molecule. Over-activation and over-expression of a specific gene/protein can co-exist, and often the existence of one contributes to the existence of the other in a cell.

Standard molecular biology techniques known in the art and not specifically described are generally followed as in Sambrook et al., Molecular Cloning: A Laboratory Manual, Cold Springs Harbor Laboratory, New York (1989, 1992), and in Ausubel et al., Current Protocols in Molecular Biology, John Wiley and Sons, Baltimore, Md. (1989). Additionally, standard methods in immunology known in the art and not specifically described are generally followed as in Stites et al. (eds), Basic and Clinical Immunology (8^{th} Edition), Appleton & Lange, Norwalk, Conn. (1994) and Mishell and Shiigi (eds), Selected Methods in Cellular Immunology, W. H. Freeman and Co., New York (1980).

The contents of all references cited herein (including journal articles, books, published applications, and patents) are hereby incorporated by reference in their entirety.

### EXAMPLES

PKCδ is Required for Survival of Cells Expressing Activated p21Ras

### Materials and Methods

Plasmids and Reagents. The activated Ras effectors, Raf-1 (Raf-22W), PI₃K (p110-CAAX), R1f (R1f-CAAX) and RalGDS (RalA-28N, dominant-negative) were cloned into the pBabe puro vector (Rodriguez-Viciana P, 1997) and the H-Ras effector loop mutants, S35, G37 and C40 (double mutations) and H-ras V12 single mutant (V12) were cloned into the pSG5 vector (Peyssonnaux C, 2000). These vectors were kindly provided by C. Counter (Duke University) and J. Downward (MRC, UK). The pEGFP-PKCδ-KR (dominant-negative PKCδ) vector (Bharti A, 1998) was kindly provided by D. Kufe (Dana-Farber Cancer Institute).

Chemical inhibitors used in this study specific to PKC isoforms, PI₃K, p21^{Ras}, and MAPK are listed in Table 3. All inhibitors were dissolved in dimethyl sulfoxide for use, and their effects were measured relative to dimethyl sulfoxide (vehicle)-treated controls. The concentration of all inhibitors was optimized to produce greater than 90% inhibition of target molecule activity.

TABLE 3
Chemical inhibitors used

| | Concentration | | |
|---|---|---|---|
| | (μM) | Target | Vendor |
| FPTIII | 100 | p21^{Ras} | Calbiochem |
| Rottlerin | 20 | PKCδ | Calbiochem |
| Ly294002 | 10 | PI₃K | Calbiochem |
| PD98095 | 10 | MAPK | Calbiochem |
| Safingol | 5 | PKCα | Calbiochem |
| Go6976 | 0.3 | PKCα/β | Calbiochem |
| Bisindolylmaleimide I | 10 | PKC β1 and β2 | Calbiochem |
| Ly333531 | 0.01 | pan-PKC | A.G. Scientific |
| HMG | 120 | pan-PKC | Calbiochem |

Cell Culture and Treatment. NIH/3T3 and Balb cell lines were obtained from ATCC (American Type Culture Collection, Rockville, Md.). NIH/3T3-Ras cells were produced by stable transfection of v-Harvey ras, and selected and maintained in 0.5 mg/ml of Geneticin (Gibco BRL, Gaithersburg, Md.). Ki-v-ras-Balb (KBalb) cells were produced by stable infection of Balb with retroviral vector stocks containing v-Kirsten ras, and were selected and maintained with Geneticin. The human pancreatic tumor lines Hs 766s, BxPC-3 and MIA PaCa-2, were obtained from ATCC. All cell lines were maintained in Dulbecco Modified Eagle medium (DMEM) (Gibco BRL) supplemented with 2 mM 1-glutamine, 100 U/ml of penicillin, and 100 μg/ml of streptomycin (Gibco BRL). Media was additionally supplemented with 10% DCS (NIH/3T3, NIH/3T3-Ha-v-Ras, Balb/3T3, Ki-v-ras-Balb, and Balb-myc), or 10% FBS (BxPC-3, MIA PaCa-2). Cells were cultured at 37° C. and 5% CO₂.
Cell Proliferation Assay. Cell proliferation was assessed using an MTT [3-(4,5-dimethylthiazol-2-yl)-2,5-diphenyltetrazolium bromide] assay (Roche, Mannheim, Germany). The number of viable cells growing in a single well on a 96-well microliter plate was estimated by adding 10 μl of MTT solution (5 mg/ml in phosphate-buffered saline [PBS]). After 4 h of incubation at 37° C., the stain was diluted with 100 μl of dimethyl sulfoxide. The optical densities were quantified at a test wavelength of 550 nm and a reference wavelength of 630 nm on a multiwell spectrophotometer.
siRNA Knockdown of PKCδ. siRNA duplexes (siRNAs) were obtained from Qiagen (Valencia, Calif.). The siRNA sequences for targeting PKCδ were PKCδsiRNA1 (5′-GAUGAAGGAGGCGCUCAGTT-3 (SEQ ID NO: 4)) and PKCδsiRNA2 (5′-GGCUGAGUUCUGGCUGGACTT-3′ (SEQ ID NO: 5)) (Yoshida K, 2003). The corresponding scrambled siRNAs were used as negative control. These siRNA sequences were also cloned into the pRNA6.1-Neo vector with a GFP tag. Transfection of siRNA was performed using 50 nM PKCδ siRNA, or the same amount of scrambled siRNA and Lipofectamine 2000 (Invitrogen, Carlsbad, Calif.), according to the manufacturer's instructions. Transfection of plasmid-based siRNA vectors was carried out using the same method. PKCδ protein levels were determined by immunoblot analysis.
Assay of PKCδ Kinase Activity. PKCδ activity was measured with an assay kit (Upstate Cell Signaling). After two days of treatment of rottlerin, cells were lysed in 50 mM Tris-HCl, pH 8.0, 150 mM NaCl, 1% Triton X-100, 20 mM MgCl₂, 5 mM EGTA, 1 mM othovanadate, 50 μg/ml PSMF and 3 μg/ml aprotinin. PKCδ was immunoprecipitated from 200 μg of protein extracts as described above. Immunocomplexes were washed three times with the kinase buffer (20 mM Tris-HCl, 10 mM MgCl2, pH 7.5) and then incubated with a PKC-specific peptide substrate, [γ-³³P]ATP, and inhibitors of cAMP-dependent kinase and calmodulin kinase for 10 min at 30° C. ³³P incorporated into the substrate was separated from residual ³³P using p81 filters and subsequently quantified by scintillation counting. β-actin antibody was used as negative control in immunoprecipitations.
p21^{Ras} Activity Assays. Cells were cultured in DMEM containing 0.5% serum for 48 hours. Then cells were lysed in a buffer containing 25 mM HEPES, pH 7.5, 150 mM NaCl, 1% Igepal CA-630, 0.25% sodium deoxycholate, 10% glycerol, and 25 mM NaF. Protein was normalized to 1 μg/μl, and activated Ras was affinity-precipitated by mixing 1 mg of cell lysate with 10 μg of Raf-RBD-agarose bead conjugate (Upstate Biotechnology, Inc.) for 60 min at 4° C. The conjugates were washed three times in lysate buffer and then separated on a 10% SDS-polyacrylamide gel. Proteins were transferred onto a polyvinylidene difluoride (PVDF) membrane and immunoblotted with a monoclonal p21^{Ras} antibody (BD Transduction Laboratories).
DNA Profile Analysis. 1×10⁵ cells were plated in 60 mm dish and grown until confluent. Cells were harvested and resuspended with 1 ml of a 35% ethanol/DMEM solution for five min at room temperature. Cells were collected and stained with solution containing 50 μg/ml RNase and 25 units/ml propidium iodide in PBS, and incubated in the dark for 30 min at room temperature for flow cytometric analysis.
Immunoblotting Analysis. Harvested cells were disrupted in a buffer containing 20 mM Tris (pH 7.4), 0.5% NP-40, and 250 mM NaCl. Total protein (40 μg) was separated on 10% SDS-polyacrylamide gels and transferred to nitrocellulose membranes or PVDF membranes. Membranes were blocked overnight and probed with affinity-purified antibodies against p21^{Ras} (BD Transduction Labs), PKCδ (BD Transduction Lab) and β-actin (Sigma). After washing, the blots were incubated with horseradish peroxidase-conjugated secondary antibodies and visualized using the Amersham enhanced chemiluminescence ECL system.
Protein Stability Assay. Cells were incubated with 100 μg cycloheximide/ml for the time periods indicated. Cell extracts were prepared as above. Proteins (100 μg per lane) were analyzed by 10% SDS-PAGE electrophoresis and immunoblotting.
Cell Apoptosis Assay. Terminal deoxynucleotidyl transferase-mediated deoxyuridine triphosphate nick end-labeling (TUNEL) assay was used for apoptosis assay. Briefly, cells were fixed with 4% paraformaldehyde in PBS overnight at 4° C. The samples were washed three times in PBS and permeabilized by 0.2% Triton X-100 in PBS for 15 min on ice. After rinsing twice with PBS, the samples were incubated with the TMR red TUNEL reagent (Roche) at 37° C. in the dark, according to the manufacturer's instructions. Apoptotic cells were identified by fluorescent microscopy.

### Results

Selective Down-Regulation of PKCδ Induces Apoptosis in Cells with Activated p21^{Ras}

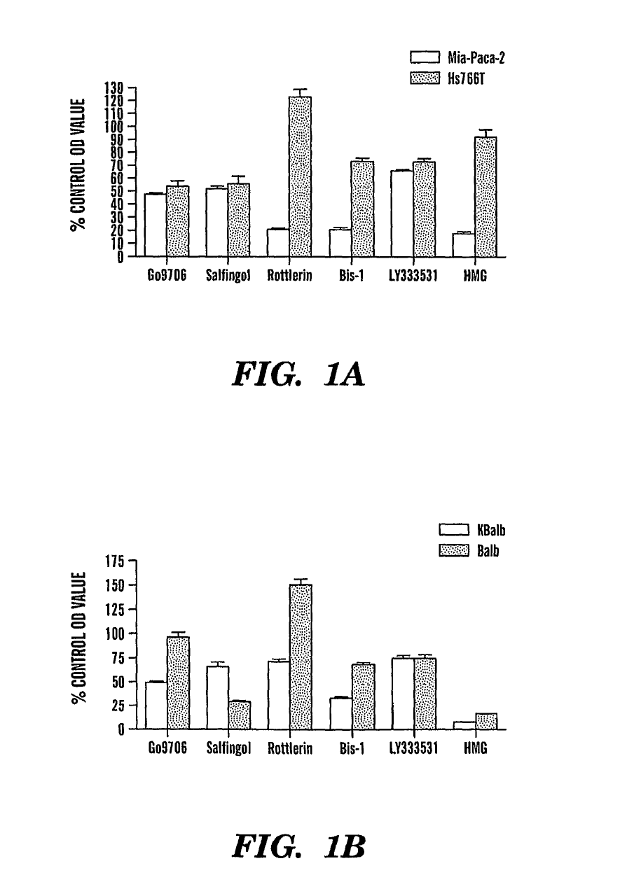

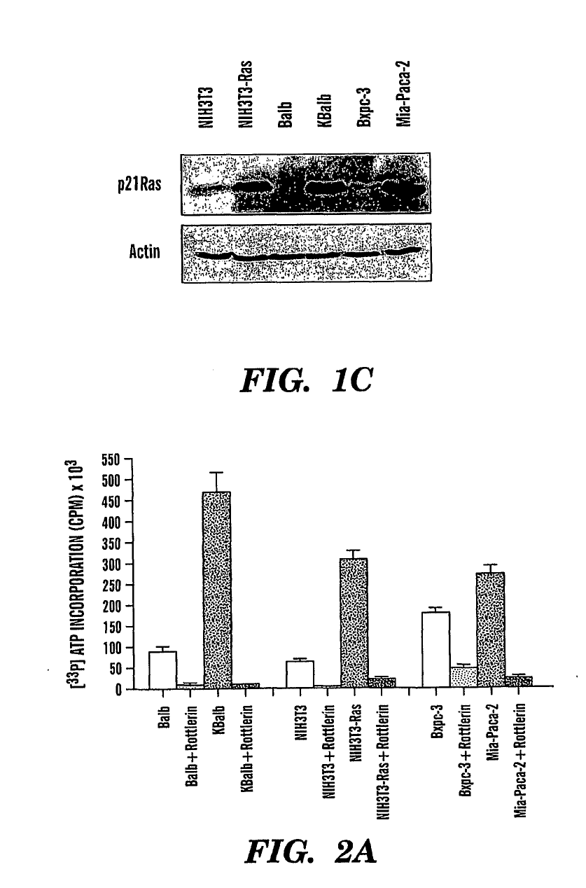

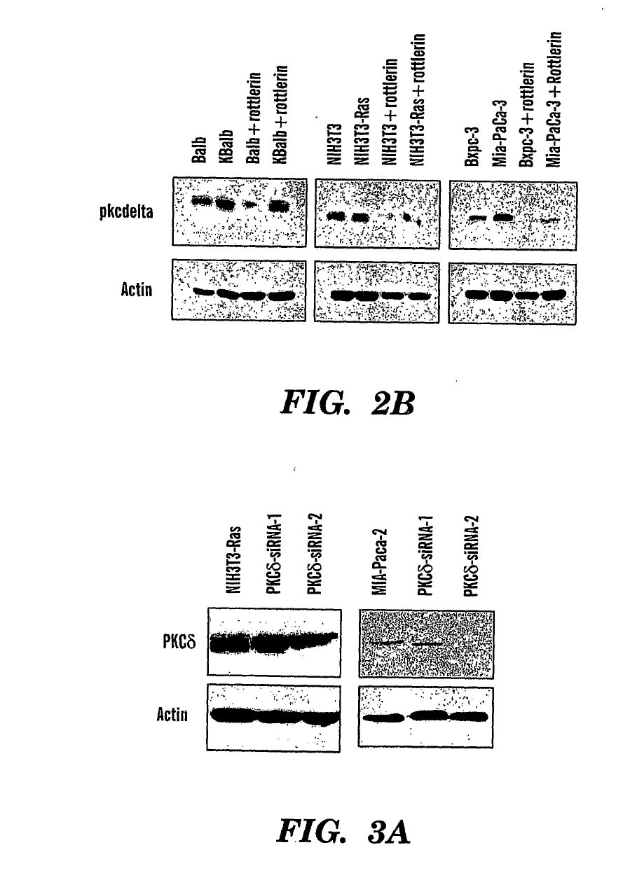

Initially, six isoform-specific or non-specific PKC inhibitors were used to suppress PKCα, PKCα/β, PKCβ1/2, PKCδ or whole PKC activity (Table 1). Optimal effective concentrations of each inhibitor were determined in pilot experiments (data not shown). Cells were treated for 48 hours and cell proliferation was detected by MTT assay. Among the isoform-specific inhibitors, rottlerin dramatically and specifically decreased the proliferation of both MIA PaCa-2 and KBalb cells (both of which express mutated p21^{Ras}), compared to the corresponding Hs766T cells and Balb cells (which contain wild-type p21^{Ras}) (FIG. 1A; 1B). The elevated activity of p21^{Ras} in the NIH/3T3-Ras cells, the KBalb cells and the MIA PaCa-2 cells was confirmed by pull-down assay (FIG. 1C). The pan-PKC activity inhibitors Bis-1 and HMG also strongly suppressed the growth of the cells containing activated p21^{Ras}, consistent with previous studies (Liou J S 2000, 2004). In contrast, rottlerin stimulated proliferation in cell lines expressing wild-type p21^{Ras} (Balb and Hs766T cells). PKCδ expression and activity was analyzed in the rottlerin-treated cells to confirm suppression of activity. After 48 hr treatment with 20 μM rottlerin, cells were lysed and PKCδ was pulled down by a specific anti-PKCδ antibody and the incorporation of [γ-³³P] ATP into a specific substrate peptide (QKRPSQRSKYL (SEQ ID NO: 6) by the precipitates was quantitated. Exposure to rottlerin blocked greater than 95% of the PKCδ activity in all of the cell lines tested except BxPC-3 (FIG. 2A). Rottlerin also suppressed PKCδ levels somewhat in all cell lines tested (FIG. 2B).

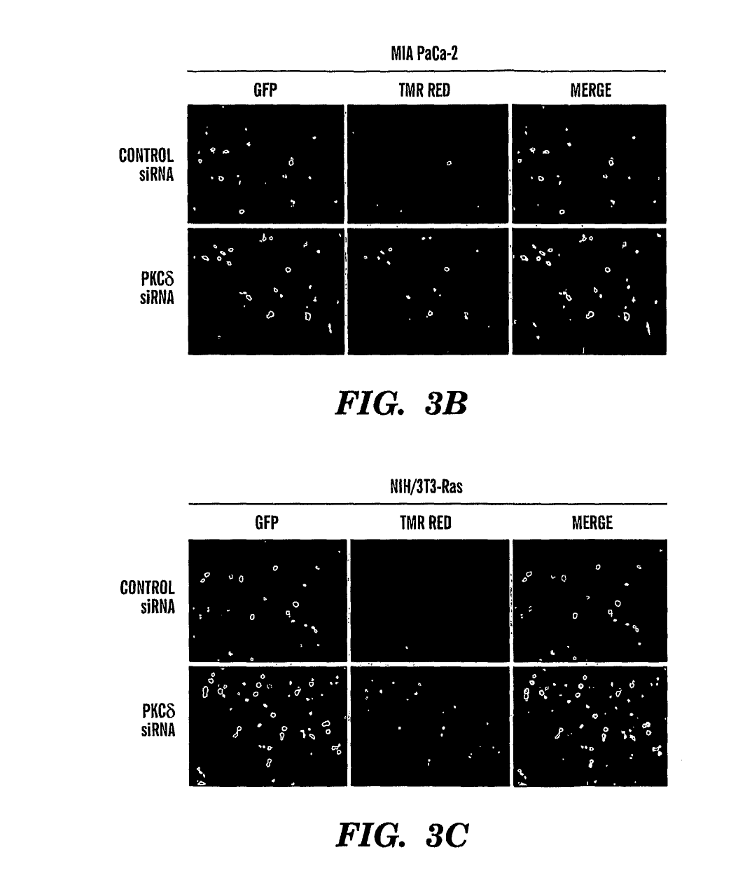

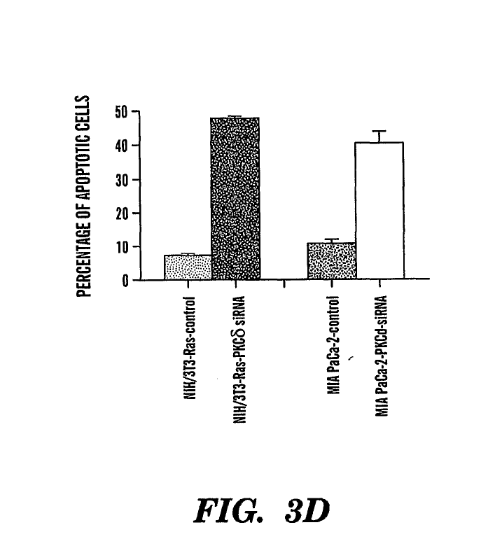

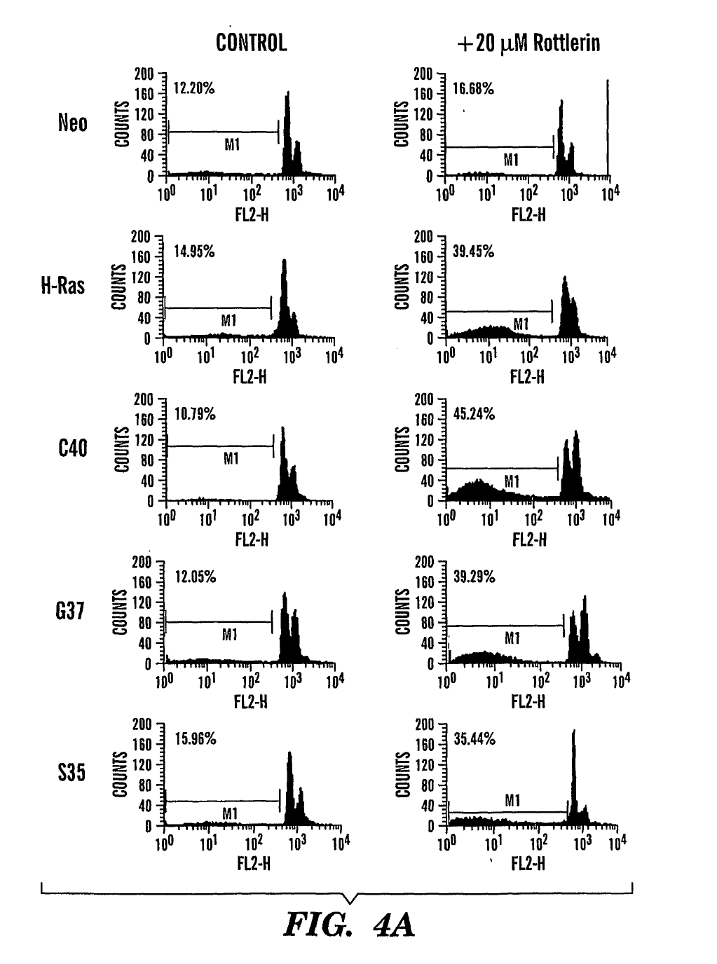

As the specificity of chemical kinase inhibitors is never absolute, two specific genetic techniques were employed to block PKCδ activity. Two PKCδ-specific siRNAs targeted at different PKCδ sequences were designed. 48 hours of transfection of MIA PaCa-2 and NIH/3T3-Ras cells, immunoblot analysis demonstrated that expression of PKCδ protein was significantly diminished by transfection with PKCδ-siRNA-2. In contrast, PKCδ-siRNA-1 did not show any knockdown of PKCδ protein (FIG. 3C). Because the efficiency of transient transfection in these cells was less than 50%, these analyses likely underestimate the activity of the siRNA oligos in an individual cell.

Seventy-two hours after transfection with pRNA-U6.1-GFP-control siRNA (scrambled siRNA) or pRNA-U6.1-GFP-PKCδ siRNA-2, MIA PaCa-2 and NIH/3T3-Ras cells were harvested to determine the apoptotic fraction by TUNEL assay. Cells which took up the vector DNA were detected by green fluorescence. TUNEL-positive cells appeared red. Superimposition displayed transfected, apoptotic cells as yellow (FIGS. 3A & B). For cells transfected with the PKCδ siRNA, 40-50% cells were undergoing apoptosis at the 48 hr time point, whereas cells transfected with the control siRNA vector displayed a frequency of apoptosis less than 10% (FIG. 3D).

Similar results were obtained when a dominant-negative, kinase-dead PKCδ was used as an alternative method of blocking PKCδ activity specifically in cells expressing an activated p21^{Ras} or wild-type p21^{Ras}. Transfection of NIH/3T3-Ras cells with the dn-PKCδ vector produced a significant fraction with a hypodiploid (apoptotic) DNA content, compared to the apoptotic fraction in cells expressing a wild-type p21^{Ras}. Transfection of the empty vector as a control generated no significant apoptosis above background levels. The induction of apoptosis by expression of PKCδ with a single-base mutation rendering it catalytically inactive also demonstrates that it is the kinase activity of PKCδ that is required for survival of cells expressing activated p21^{Ras}, rather than some other, undefined function of the molecule.

### PI₃K Ras Effector Pathway is Sufficient to Sensitize Cells to Apoptosis by PKCδ Inhibition

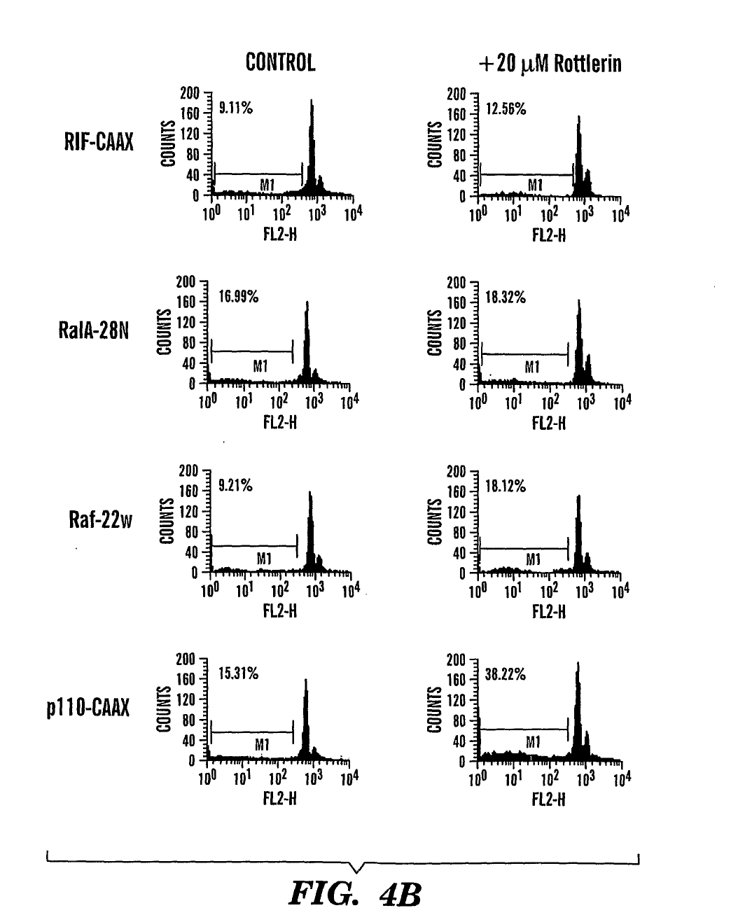

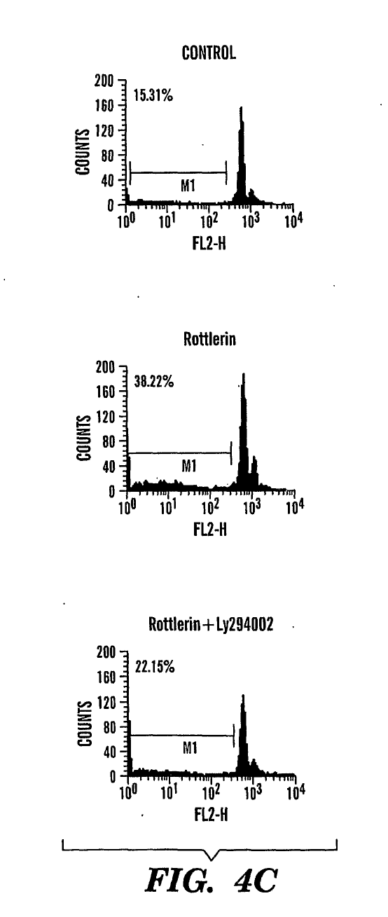

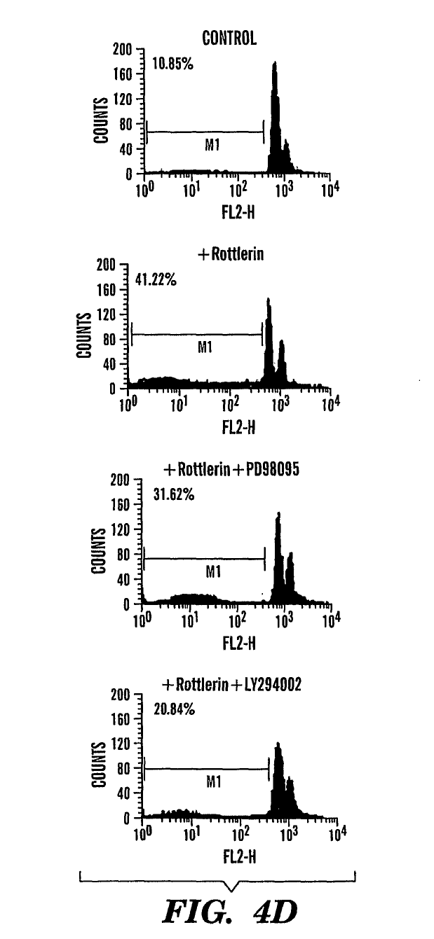

Although previous studies have demonstrated that either constitutive expression of activated p21^{Ras} or acute increases in endogenous p21^{Ras} activity stimulate apoptosis following inhibition of PKC activity in multiple types of cells, the roles of specific Ras downstream effectors in the process have never been determined. In general, three effector pathways activated by Ras have been defined: Raf1/MAPK, Ras.GDS/Ras/Rho, and PI₃K. To begin to address this question, p21^{Ras} effector mutants, consisting of the activating Ras mutation (V12) and a second mutation (S35, G37, or C40) were employed. The three RasV12 mutants (S35, G37, and C40) differ by their ability to bind to p21^{Ras} effectors (Raf, Ral-GEFs, and the p110 subunit of PI₃K, respectively). Cells were treated with 20 μM rottlerin for 60 h, and subjected to flow cytometric analysis. All three p21^{Ras} downstream effector mutants stimulated apoptosis to some extent after inhibition of PKCδ, although the C40 mutant consistently generated the greatest amount of apoptosis (FIG. 4A).

The Ras-effector mutants are not completely specific in their activation of a single Ras effector pathway, and each activates all three pathways to some degree. In order to more clearly identify the down-stream effector of p21^{Ras} relevant to Ras-mediated apoptosis, single effector pathways were activated using expression vectors for activated PI₃K (p110-CAAX), Raf (Raf-22W), and Ral-GEF (RIF-CAAX) into NIH/3T3 cells. The dominant-negative RalA-22N vector was used as control. Constitutive activation of the PI₃K (p110-CAAX) was capable of inducing apoptosis after PKCδ inhibition (apoptotic frequency: 38.22%). In contrast, the other Ras effectors Raf and Ral-GEF induced little apoptosis in response to PKCδ suppression (FIG. 4B).

To further confirm that the downstream p21^{Ras} effector PI₃K plays a critical role in Ras-mediated apoptosis, a PI₃K specific inhibitor (LY294002) was employed. A NIH/3T3 cell line stably-expressing p110-CAAX was pre-treated with Ly294002 (20 μM) for 30 min, and then rottlerin (20 μM) was added for 60 hours. The results are shown in. In the presence of the PI₃K inhibitor, the apoptosis induced by rottlerin decreased nearly 50% (FIG. 4C). Similarly, in KBalb cells, LY294002 blocked 50% of the apoptosis induced by PKCδ inhibition (FIG. 4A). In cells which contained an activated p21^{Ras}, but not those with activation of the single PI₃K-effector pathway, the MAPK inhibitor PD98095 also suppressed p21^{Ras}-dependent apoptosis to some extent, but to a substantially lesser degree than did PI₃K-inhibition. Collectively, these data demonstrate that Ras-mediated apoptosis by PKCδ inhibition is mediated through the downstream effector PI₃K. In addition the MAPK effector pathway also contributes significantly to the process.

p21^{Ras} Up-Regulates PKCδ Protein Levels by Stabilization

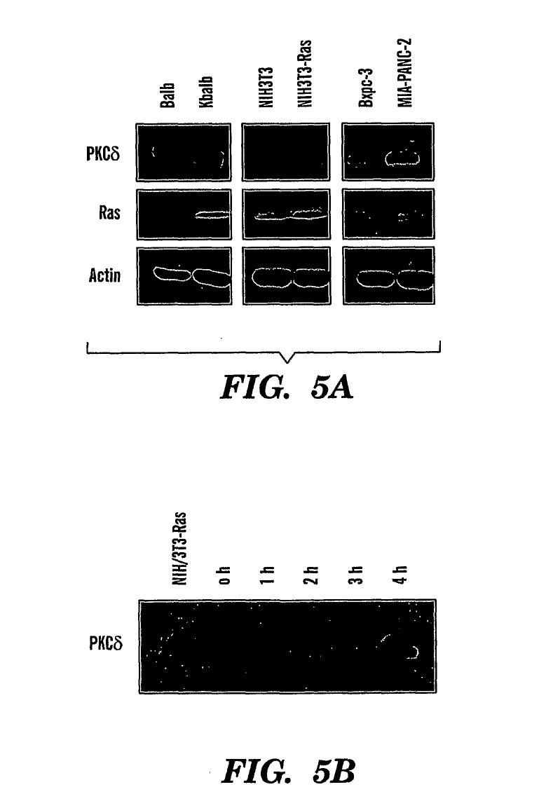

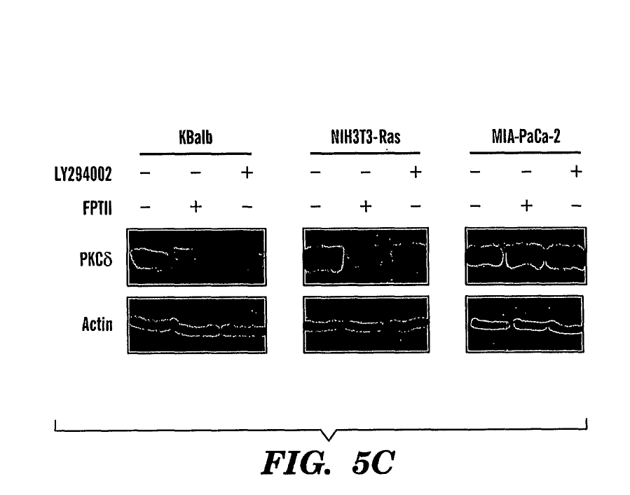

Cell lines expressing an activated p21^{Ras} invariably expressed substantially more PKCδ protein than did their wild-type p21^{Ras}-expressing counterparts (FIG. 5A), suggesting that p21^{Ras} activity may up-regulate PKCδ expression. To test this hypothesis, NIH/3T3 cells were transfected with pSG5-H-(V12)Ras and PKCδ protein levels assayed over time. PKCδ increased rapidly after transfection, reaching peak levels at 4 hours (FIG. 5B). In contrast, transfection with the empty pSG5 vector had no effect on PKCδ levels. Conversely, when p21^{Ras} or PI₃K activity in KBalb and NIH/3T3-Ras cells was inhibited by the Ras inhibitor FPTII or the PI3K inhibitor LY294002, PKCδ protein levels fell (FIG. 5C).

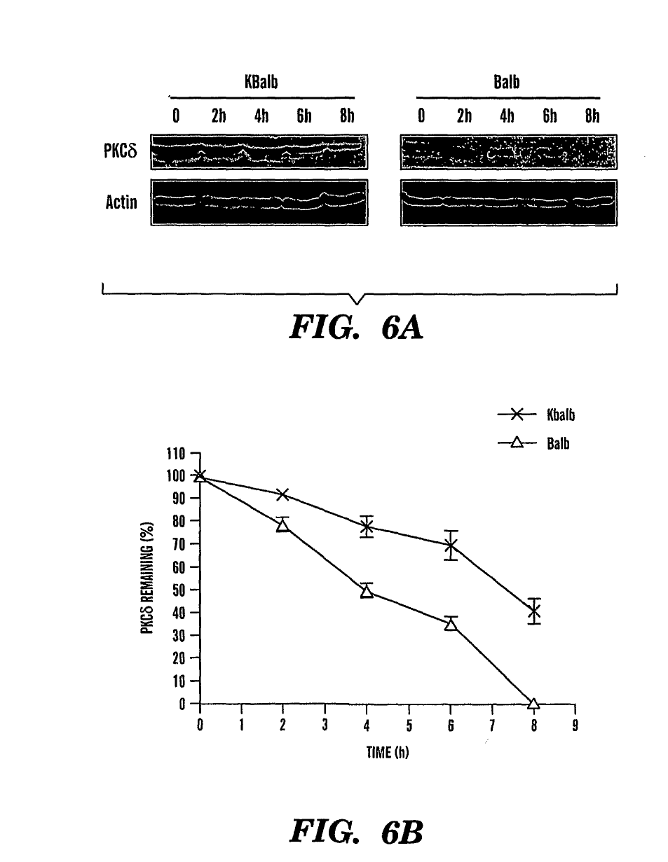

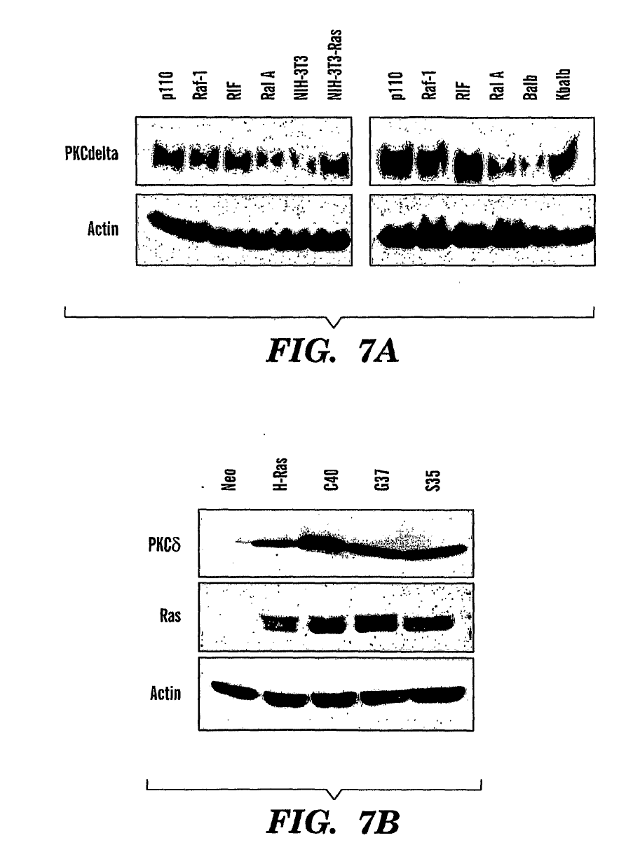

The regulation of PKCδ by p21^{Ras} is not at the level of transcription, as PKCδ transcript levels, assessed by quantitative RT-PCR, did not vary as a function of p21^{Ras} activity. To determine if p21^{Ras} protein affects PKCδ protein stability, the half-life of PKCδ protein was examined in matched cell lines containing either wild-type or activated PKCδ. The half-life of PKCδ was prolonged at least 2-fold in cells expressing an activated p21^{Ras} (FIGS. 6A & B).

To determine which p21Ras effector pathway(s) mediated the upregulation of PKCδ, p110-CAAX, Raf-22w, RIF-CAAX, and RalA-28N were stably transfected into two cell lines, and PKCδ protein was quantitated (FIG. 6A). The p110-CAAX was consistently the most potent inducer of PKCδ expression in both Balb and NIH/3T3 cells, although each of the other two effector pathways could also induce PKCδ expression to a variable extent. Similarly, the C40 p21Ras-effector loop mutant, which activated predominantly the PI3K pathway, was the most potent of the effector mutants for induction of PKCδ (FIG. 6B).

### Discussion

The p21^{Ras} family members comprise critical molecular switches transducing signals to diverse downstream pathways, ultimately controlling such processes as proliferation, cytoskeletal integrity, apoptosis, cell adhesion, and cell migration (Cowley S, 1994; Stice L L, 2002; Nakada M, 2005; Rajalingam K, 2005). p21^{Ras} and Ras-related proteins are frequently deregulated in cancers, leading to increased invasion and metastasis and enhancement of survival by activation of anti-apoptotic pathways. Paradoxically, several studies have demonstrated that enforced, high-level expression of oncogenic p21^{Ras} can induce a permanent growth arrest in normal cells, mimicking natural senescence, (Serrano M, 1997; Lin A W, 1998). Activation of the Raf-1/MEK/p38MAPK pathway is thought to be essential for oncogenic p21^{Ras}-induced senescence (Wang, 2002, and Lin, 1998). Additionally, inactivation of the ARFp53 tumor suppressor pathway in mouse fibroblasts and skin keratinocytes, or inactivation of the p16Rb tumor suppressor pathway in human fibroblasts, can bypass p21^{Ras}-induced senescence, suggesting that cellular fate resulting from oncogenic p21^{Ras} signaling is dependent upon the cellular context and the integration of tumor suppressor signals (Brookes, 2002; Serrano, 1997). Various laboratories have established p21^{Ras} as a modulator of apoptosis in transformed cells and malignant cells, and in normal cells. Through FADD, caspase 8, and downstream effector reactive oxygen species (ROS), p21^{Ras} sensitizes cells to apoptosis induced by PKC inhibition (Liou, 2000; Chen, 1998; Chen, 2001). As different PKC isozymes may have opposing functions with respect to cellular proliferation, differentiation and apoptosis, a more complete understanding of this process required identification of the specific PKC isozyme required for survival of cells expressing activated p21^{Ras}, along with elucidation of the specific p21^{Ras} downstream effectors evoking the apoptotic outcome.

Senescence as the outcome of enforced p21^{Ras} activity is observed predominantly in primary cells (Sebastian T, 2005; Tombor B, 2003; Huot T J, 2002; Pearson M, 2000). Cellular senescence is characterized by loss of proliferative potential, resulting from repeated rounds of replication. Senescence is dependent upon the integration of a number of pathways that together result in a permanent and irreversible blockade of cell cycle progression. Currently, three signaling pathways have been reported to contribute to a senescent fate: p16^{INK4a}/Rb pathway, p19^{ARF}/p53/p21^{cip1} and PTEN/p27^{Kip1} (Brmgold F, 2000). Primary mammalian cells are characteristically refractory to neoplastic transformation by p21^{Ras}. In contrast, in immortal mammalian cell lines, which are deficient in one or more of these pathways, unregulated p21^{Ras} expression may lead to transformation rather than senescence. Interestingly, certain cyclin-dependent kinases (CDKs) may be required for both the senescent and the transformed fate. Primary murine fibroblasts deficient in p16^{INK4a}/p19^{ARF}, p19^{ARF} or p53 can be transformed by p21^{Ras}, but cells deficient in p21^{Cip1} and p27^{Kip1} cannot be transformed (Serrano 1997; and Pantoia, 1999). Among the various Ras downstream effectors, the critical one for induction of senescence appears to be the Raf/MEK/MAPK cascade, suggesting that p21^{Ras}-driven proliferation is the relevant stimulus that triggers senescence (Lin, 1998).

Apoptosis represents a third possible fate for a cell with dysregulated expression of p21^{Ras}. In this case, the relevant downstream effector of p21^{Ras} is the PI₃K pathway. Studies described herein demonstrate that PKCδ activity is required to prevent the induction of apoptosis in cells expressing activated p21^{Ras}. It is noteworthy that p21^{Ras} activity, and in particular activation of the PI₃K pathway, upregulates PKCδ protein levels, thus positively reinforcing an anti-apoptotic, protective response to p21^{Ras} dysregulation in the cell. Conversely, when this induction was prevented by siRNA knockdown of PKCδ, programmed cell death was initiated.

PKCδ has been reported to both positively and negatively regulate apoptotic programs (Watanabe T 1992; Mischak H, 1993; Liao L, 1994; Li w, 1998; Pal S, 1997). These findings have generated conflicting hypotheses as to the role of PKCδ in the control of cell proliferation and survival. The normal phenotype of PKCδ-null mice demonstrates that PKCδ is not required for appropriate control of cell proliferation during normal development (Leitges M, 2001; Miyamoto A 2002). In contrast, PKCδ may be suborned during cellular transformation and become necessary for one or more components of the malignant phenotype. Inhibition of PKCδ was reported to inhibit the metastatic potential of breast cancer cells (Kiley S C, 1999) and to reduce their survival (McCrachen M A, 2003). Similar results were reported using non-small cell lung cancer cells (Clark A S, 2003). The findings reported herein support this hypothesis. The results obtained from the experiments reported herein indicate that PKCδ functions as a survival signal in a variety of cells with dysregulated activation of p21^{Ras}. PKCδ expression is upregulated in response to p21^{Ras} activity, primarily through PI₃K activation, and is required for the survival of these cells. However, PKCδ is not required for the survival or proliferation of the non-transformed counterparts of these cells, and indeed suppression of PKCδ actually leads to a small but reproducible increase in their proliferation.

This work shows how p21^{Ras}-mediated apoptosis can be caused by molecules required to redirect p21^{Ras} signaling from a proliferative/transforming outcome to an apoptotic fate. Furthermore, elucidation of the particular PKC isoform necessary for survival of cells transformed by p21^{Ras} demonstrates that selective suppression of PKCδ activity and the consequent induction of apoptosis is a novel strategy for targeting of tumor cells containing an aberrantly increased Ras signaling.

### REFERENCES

1. Abbas T, White D, Hui L, Yoshida K, Foster D A, Bargonetti J. Inhibition of human p53 basal transcription by down-regulation of protein kinase C delta. J Biol Chem. 2004 Mar. 12; 279(11):9970-7.
2. Adiei A A. Blocking oncogenic Ras signaling for cancer therapy. J Natl Cancer Inst. 2001 July 18; 93(14):1062-74. Review.
3. Basu A. Involvement of protein kinase C-delta in DNA damage-induced apoptosis. J Cell Mol Med. 2003 October-December; 7(4):341-50.
4. Bharti A, Kraeft S K, Gounder M, Pandey P, Jin S, Yuan Z M, Lees-Miller S P, Weichselbaum R, Weaver D, Chen L B, Kufe D, Kharbanda S. Inactivation of DNA-dependent protein kinase by protein kinase C delta: implications for apoptosis. Mol Cell Biol. 1998 November; 18(11):6719-28.
5. Bringold F, Serrano M. Tumor suppressors and oncogenes in cellular senescence. Exp Gerontol. 2000 May; 35(3):317-29.
6. Brodie C, Blumberg P M. Regulation of cell apoptosis by protein kinase c delta. Apoptosis. 2003 January; 8(1):19-27.
7. Brookes S, Rowe J, Ruas M, Llanos S, Clark P A, Lomax M, James M C, Vatcheva R, Bates S. Vousden K H, Parry D, Gruis N, Smit N, Bergman W, Peters G. INK4a-deficient human diploid fibroblasts are resistant to RAS-induced senescence. EMBO J. 2002 Jun. 17; 21(12):2936-45.
8. Chen C Y, Faller D V. Phosphorylation of Bcl-2 protein and association with p21Ras in Ras-induced apoptosis. J Biol Chem. 1996 Feb. 2; 271(5):2376-9.
9. Chen C Y, Juo P, Liou J S, Li C Q, Yu Q, Blenis J, Faller D V. The recruitment of Fas-associated death domain/caspase-8 in Ras-induced apoptosis. Cell Growth Differ. 2001 June; 12(6):297-306.
10. Chen C Y, Liou J, Forman L W, Faller D V. Correlation of genetic instability and apoptosis in the presence of oncogenic Ki-Ras. Cell Death Differ. 1998 November; 5(11):984-95.
11. Clark A S, West K A, Blumberg P M, Dennis P A. Altered protein kinase C (PKC) isoforms in non-small cell lung cancer cells: PKCdelta promotes cellular survival and chemotherapeutic resistance. Cancer Res. 2003 Feb. 15; 63(4):780-6.
12. Cowley S, Paterson H, Kemp P, Marshall C J. Activation of MAP kinase kinase is necessary and sufficient for PC12 differentiation and for transformation of NIH 3T3 cells. Cell. 1994 Jun. 17; 77(6):841-52.
13. Downward J. Targeting RAS signalling pathways in cancer therapy. Nat Rev Cancer. 2003 January; 3(1):11-22. Review.
14. Ghayur T, hugunin M, Talanian R V, Ratnofsky S, Quinlan C, Emoto Y, Pandey P, Datta R, Huang Y, Kharbanda S, Allen H, Kamen R, Wong W, Kufe D. Proteolytic activation of protein kinase C delta by an ICE/CED 3-like protease induces characteristics of apoptosis. J Exp Med. 1996 Dec. 1; 184(6):2399-404.
15. Hiraragi H, Michael B, Nair A, Silic-Benussi M, Ciminale V, Lairmore M. Human T-lymphotropic virus type 1 mitochondrion-localizing protein p13II sensitizes Jurkat T cells to Ras-mediated apoptosis. J. Virol. 2005, 79(15): 9449-57.
16. Huot T J, Rowe J, Harland M, Drayton S, Brookes S, Gooptu C, Purkis P, Fried M, Bataille V, Hara E, Newton-Bishop J, Peters G. Biallelic mutations in p16(INK4a) confer resistance to Ras- and Ets-induced senescence in human diploid fibroblasts. Mol Cell Biol. 2002 December; 22(23):8135-43.
17. Jaken S, Parker P J. Protein kinase C binding partners. Bioessays. 2000 March; 22(3):245-54.
18. Johnson C l, Lu D, huang J, Basu A. Regulation of p53 stabilization by DNA damage and protein kinase C. Mol Cancer Ther. 2002 August; 1(10):861-7.
19. Jung J W, Cho S D, Ahn N S, Yang S R, Park J S, Jo E H, Hwang J W, Jung J Ym Kim S H, Kang K S, Lee Y S. Ras/MAP kinase pathways are involved in Ras specific apoptosis induced by sodium butyrate. Cancer Lett. 2005 Jul. 28; 225(2):199-206.
20. Kiley S C, Clark K J, Goodnough M, Welch D R, Jaken S. Protein kinase C delta involvement in mammary tumor cell metastasis. Cancer Res. 1999 Jul. 1; 59(13):3230-8.
21. Klampfer L, Swaby L A, Huang J, Sasazuki T, Shirasawa S, Augenlicht L. Oncogenic Ras increases sensitivity of colon cancer cells to 5-FU-induced apoptosis. Oncogene. 2005 Jun. 2; 24(24):3932-41.
22. Leitges M, Mayr M, Braun U, Mayr, Li C, Pfister G, Ghaffari-Tabrizi N, Baier G, Hu Y, Xu Q. Exacerbated vein graft arteriosclerosis in protein kinase Cdelta-null mice. J Clin Invest. 2001 November; 108(10):1505-12.
23. Li W, Jiang Y X, Zhang J, Soon L, Flechner L, Kapoor V, Pierce J H, Wang L H. Protein kinase C-delta is an important signaling molecule in insulin-like growth factor I receptor-mediated cell transformation. Mol Cell Biol. 1998 October; 18(10):5888-98.
24. Liao L, Ramsay K, Jaken S. Protein kinase C isozymes in progressively transformed rat embryo fibroblasts. Cell Growth Differ. 1994 November; 5(11): 1185-94.
25. Lin A W, Barradas M, Storie J C, van Aelst L, Serrano M, Lowe S W. Premature senescence involving p53 and p16 is activated in response to constitutive MEK/MAPK mitogenic signaling. Genes Dev. 1998 Oct. 1; 12(19):3008-19.
26. Liou J S, Chen C Y, Chen J S, Faller D V. Oncogenic ras mediates apoptosis in response to protein kinase C inhibition through the generation of reactive oxygen species. J Biol Chem. 2000 Dec. 15; 275(50):39001-11.
27. Liou J S, Chen J S, Faller D V. Characterization of p21 Ras-mediated apoptosis induced by protein kinase C inhibition and application to human tumor cell lines. J Cell Physiol. 2004 February; 198(2):277-94.
28. Ly D H, Lockhart D J, Lerner R A, Schultz P G. Mitotic misregulation and human aging. Science. 2000 Mar. 31; 287(5462):2486-92.
29. Majumder P K, Pandey P, Sun X, Cheng K, Datta R, Saxena S, Kharbanda S, Kufe D. Mitochondrial translocation of protein kinase C delta in phorbol ester-induced cytochrome c release and apoptosis. J Biol Chem. 2000 Jul. 21; 275(29):21793-6.
30. McCracken M A, Miraglia L J, McKay R A, Strobl J S. Protein kinase C delta is a prosurvival factor in human breast tumor cell lines. Mol Cancer Ther. 2003 March; 2(3):273-81.
31. Mecklenbrauker I, Kalled S L, Leitges M, Machay F, Tarakhovsky A. Regulation of B-cell survival by BAFF-dependent PKCdelta-mediated nuclear signalling. Nature. 2004 Sep. 23; 431(7007):456-61.
32. Mecklenbrauker I, Saijo K, Zheng N Y, Leitges M, Tarakhovsky A. Protein kinase Cdelta controls self-antigen-induced B-cell tolerance. Nature. 2002 Apr. 25; 416(6883):860-5.
33. Mischak H, Pierce J H, Goodnight J, Kazanietz M G, Blumberg P M, Mushinski J F. Phorbol ester-induced myeloid differentiation is mediated by protein kinase C-alpha and -delta and not by protein kinase C-beta II, -epsilon, -zeta, and -eta. J Biol Chem. 1993 Sep. 25; 268(27):20110-5.
34. Miyamoto A, Nakayama K, Irnaki H, Hirose S, Jiang Y, Abe M, Tsukiyama T, Nagahama H, Ohno S, Hatakeyama S, Nakayama K I. Increased proliferation of B cells and auto-immunity in mice lacking protein kinase Cdelta. Nature. 2002 Apr. 25; 416(6883):865-9.
35. Nakada M, Niska J A, Tran N L, McDonough W S, Berens M E. EphB2/R-Ras signaling regulates glioma cell adhesion, growth, and invasion. Am J Pathol. 2005 August; 167(2):565-76.
36. Pal S, Claffey K P, Dvorak H F, Mukhopadhyay D. The von Hippel-Lindau gene product inhibits vascular permeability factor/vascular endothelial growth factor expression in renal cell carcinoma by blocking protein kinase C pathways. J Biol Chem. 1997 Oct. 31; 272(44):27509-12.
37. Panto Ja C, Serrano M. Murine fibroblasts lacking p21 undergo senescence and are resistant to transformation by oncogenic Ras. Oncogene. 1999 Sep. 2; 18(35):4974-82.
38. Parker P J, Murray-Rust J. PKC at a glance. J Cell Sci. 2004 Jan. 15; 117(Pt 2):131-2. Review.
39. Pearson M, Carbone R, Sebastiani C, Cioce M, Fagioli M, Saito S, Higashimoto Y, Appella E, Minucci S, Pandolfi P P, Pelicci P G. PML regulates p53 acetylation and premature senescence induced by oncogenic Ras. Nature. 2000 Jul. 13; 406(6792):207-10.
40. Pevssonnaux C, Provot S, Felder-Schmittbuhl M P, Calothy G, Eychene A. Induction of postmitotic neuroretina cell proliferation by distinct Ras downstream signaling pathways. Mol Cell Biol. 2000 October; 20(19):7068-79.
41. Rajalingam K, Wunder C, Brinkmann V, Churin Y, Hekman M, Sievers C, Rapp U R, Rudel T. Prohibitin is required for Ras-induced Raf-MEK-ERK activation and epithelial cell migration. Nat Cell Biol. 2005 August; 7(8):837-43.
42. Rodriguez-Viciana P, Warne P H, Khwaja A, Marte B M, Pappin D, Das P, Waterfield M D, Ridley A, Downward J. Role of phosphoinositide 3-OH kinase in cell transformation and control of the actin cytoskeleton by Ras. Cell. 1997 May 2; 89(3):457-67.
43. Santiago-Walker A E, Fikaris A J, Kao G D, Brown E J, Kazanietz M G, Meinkoth J L. Protein kinase C delta stimulates apoptosis by initiating G1 phase cell cycle progression and S phase arrest. J Biol Chem. 2005 Sep. 16; 280(37):32107-14.
44. Sebastian T, Malik R, Thomas S, Sage J, Johnson P F. C/EBPbeta cooperates with RB:E2F to implement Ras(V12)-induced cellular senescence. EMBO J. 2005 Sep. 21; 24(18):3301-12.
45. Serrano M, Lin A W, McCurrach M E, Beach D, Lowe S W. Oncogenic ras provokes premature cell senescence associated with accumulation of p53 and p16INK4a. Cell. 1997 Mar. 7; 88(5):593-602.
46. Sherr C J, Roberts J M. CDK inhibitors: positive and negative regulators of G1-phase progression. Genes Dev. 1999 Jun. 15; 13(12):1501-12.
47. Sherwood S W, Rush D, Ellsworth J L, Schimke R T. Defining cellular senescence in IMR-90 cells: a flow cytometric analysis. Proc Natl Acad Sci USA. 1988 December; 85(23):9086-90.
48. Stice L L, Forman L W, Hahn C S, Faller D V. Desensitization of the PDGFbeta receptor by modulation of the cytoskeleton: the role of p21 (Ras) and Rho family GTPases. Exp Cell Res. 2002 Apr. 15; 275(1):17-30.
49. Sun X, Wu F, Datta R, Kharbanda S, Kufe D. Interaction between protein kinase C delta and the c-Ab1 tyrosine kinase in the cellular response to oxidative stress. J Biol Chem. 2000 Mar. 17; 275(11):7470-3.
50. Thimmaiah K N, Easton J, Huang S, Veverka K A, Germain G S, Harwood F C, Houghton P J. Insulin-like growth factor I-mediated protection from rapamycin-induced apoptosis is independent of Ras-Erk1-Erk2 and phosphatidylinositol 3′-kinase-Akt signaling pathways. J. Biol. Chem, 2000. 275(30):22916-24
51. Shao J, Sheng H, DuBois R N, Beauchamp R D. Oncogenic Ras-mediated cell growth arrest and apoptosis are associated with increased ubiquitin-dependent cyclin D1 degradation. J Biol Chem. 2000 Jul. 28; 275(30):22916-24.
52. Tombor B, Rundell K, Oltvai Z N. Bcl-2 promotes premature senescence induced by oncogenic Ras. Biochem Biophys Res Commun. 2003 Apr. 11; 303(3):800-7.
53. Wang W, Chen J X, Liao R, Deng Q, Zhou J J, Huang S, Sun P. Sequential activation of the MEK-extracellular signal-regulated kinase and MKK3/6-p38 mitogen-activated protein kinase pathways mediates oncogenic ras-induced premature senescence. Mol Cell Biol. 2002 May; 22(10):3389-403.
54. Watanabe T, Ono Y, Taniyama Y, Hazama K, Igarashi K, Ogita K, Kikkawa U, Nishizuka Y. Cell division arrest induced by phorbol ester in CHO cells overexpressing protein kinase C-delta subspecies. Proc Natl Acad Sci USA. 1992 Nov. 1; 89(21):10159-63.
55. Wiesmuller L, Wittinghofer F. Signal transduction pathways involving Ras. Mini review. Cell Signal. 1994 March; 6(3):247-67.
56. Yoshida K, Wang H G, Miki Y, Kufe D. Protein kinase Cdelta is responsible for constitutive and DNA damage-induced phosphorylation of Rad9. EMBO J. 2003 Mar. 17; 22(6):1431-41.
57. Zhang J, Liu N, Zhang J, Liu S, Liu Y, Zhang D. PKCdelta protects human breast tumor MCF-7 cells against tumor necrosis factor-related apoptosis-inducing ligand-mediated apoptosis. J Cell Biochem. 2005 Oct. 15; 96(3):522-32.
58. Chou C k, Liang K H, Tzeng C C, Huang G C, Chuang J I, Chang T Y, Liu H S. Dominant-negative Rac1 suppresses Ras-induced apoptosis possibly through activation of NFkappaB in Ha-ras oncogene-transformed NIH/3T3 cells. Life Sci. 2005 Nov. 4.

1. A method for treating a subject with, or at risk for, developing abnormally proliferating cells resulting from aberrantly increased Ras signaling, comprising
a) determining whether a subject contains cells which have aberrantly increased Ras signaling; and
b) administering an effective amount of an agent that selectively inhibits Protein Kinase C (PKC) delta to the subject having aberrantly increased Ras signaling.

2. The method of claim 1, wherein determining step a) is by assaying for expression of activated Ras, wherein detection of such expression indicates the presence of cells with aberrantly increased Ras signaling.

3. The method of claim 2, wherein the aberrantly increased Ras signaling is determined by detection of an activated Ras protein or a nucleotide sequence encoding an activated form of Ras protein.

4. The method of claim 1, wherein the increased Ras signaling is associated with activation of a pathway selected from the group consisting of Raf1/MAPK, RasGDS/Ras/Rho, PI3K, and combinations thereof.

5. The method of claim 2, wherein the activated Ras results from a mutation in the nucleotide sequence corresponding to codon 12, 13, 59, 61, 63, 116, 117, or 146, or any combination thereof, of SEQ ID NO: 1, 2, or 3.

6. The method of claim 2, wherein the activated form of Ras is selected from the group consisting of K-ras, H-ras, and N-ras.

7. The method of claim 1, wherein the aberrantly increased Ras signaling results from over-expression of wild-type Ras.

8. The method of claim 1, wherein determining step a) is by assaying for increased activation of one or more effector pathways downstream of Ras wherein detection of such increased activation indicates the presence of cells with aberrantly increased Ras signaling

9. The method of claim 1, wherein the abnormally proliferating cell is a malignant cell.

10. The method of claim 9, wherein the malignant cell is selected from the group of cells consisting of pancreatic cancer, colorectal cancer, non-melanoma skin cancer, hematopoietic neoplasm of myeloid origin, gastrointestinal cancer, prostate cancer, ovarian cancer, breast cancer, head and neck cancer, lung cancer, non-small cell lung cancer, cancer of the nervous system, kidney cancer, retinal cancer, skin cancer, stomach cancer, liver cancer, pancreatic cancer, genital-urinary cancer, prostate cancer, colorectal cancer and bladder cancer.

11. The method of claim 9, wherein the agent inhibits PKC-delta gene expression, reduces PKC-delta protein levels, and/or inhibits kinase activity.

12. The method of claim 11, wherein the agent is selected from the group consisting of a small molecule inhibitor, a competitive inhibitor, an irreversible inhibitor, a nucleic acid, an antibody, an antibody fragment, an aptamer, and combinations there.

13. The method of claim 11, wherein the agent is selected from the group consisting of Rottlerin, Balanol, balanol analogs, KAI-9803, and combinations thereof.

14.-26. (canceled)


SEQUENCE LISTING (6 sequences)

SEQ ID NO: 1 - DNA, 1061 bp, Homo sapiens
tgccctgcgcccgcaacccgagccgcacccgccgcggacggagcccatgcgcggggcgaaccgcgcgcccccgcccccgccccgccccggcctcggccccggccctggccccgggggcagtcgcgcctgtgaacggtggggcaggagaccctgtaggaggaccccgggccgcaggcccctgaggagcgatgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctgacgcagcacaagctcaggacatggaggtgccggatgcaggaaggaggtgcagacggaaggaggaggaaggaaggacggaagcaaggaaggaaggaagggctgctggagcccagtcaccccgggaccgtgggccgaggtgactgcagaccctcccagggaggctgtgcacagactgtcttgaacatcccaaatgccaccggaaccccagcccttagctcccctcccaggcctctgtgggcccttgtcgggcacagatgggatcacagtaaattattggatggtcttgaaaaaaaaaaaaaaaaaa

SEQ ID NO: 2 - DNA, 5312 bp, Homo sapiens
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 3 - DNA, 1963 bp, Homo sapiens
gaaacgtcccgtgtgggaggggcgggtctgggtgcggctgccgcatgactcgtggttcggaggcccacgtggccggggcggggactcaggcgcctggcagccgactgattacgtagcgggcggggccggaagtgccgctccttggtgggggctgttcatggcggttccggggtctccaacatttttcccggtctgtggtcctaaatctgtccaaagcagaggcagtggagcttgaggttcttgctggtgtgaaatgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaacaagatacttttaaagttttgtcagaaaagagccactttcaagctgcactgacaccctggtcctgacttcctggaggagaagtattcctgttgctgtcttcagtctcacagagaagctcctgctacttccccagctctcagtagtttagtacaataatctctatttgagaagttctcagaataactacctcctcacttggctgtctgaccagagaatgcacctcttgttactccctgttatttttctgccctgggttcttccacagcacaaacacacctcaacacacctctgccaccccaggtttttcatctgaaaagcagttcatgtctgaaacagagaaccaaaccgcaaacgtgaaattctattgaaaacagtgtcttgagctctaaagtagcaactgctggtgattttttttttctttttactgttgaacttagaactatgcctaatttttggagaaatgtcataaattactgttttgccaagaatatagttattattgctgtttggtttgtttataatgttatcggctctattctctaaactggcatctgctctagattcataaatacaaaaatgaatactgaattttgagtctatcctagtcttcacaactttgacgtaattaaatccaacttttcacagtgaagtgcctttttcctagaagtggtttgtagactcctttataatatttcagtggaatagatgtctcaaaaatccttatgcatgaaatgaatgtctgagatacgtctgtgacttatctaccattgaaggaaagctatatctatttgagagcagatgccattttgtacatgtatgaaattggttttccagaggcctgttttggggctttcccaggagaaagatgaaactgaaagcatatgaataatttcacttaataatttttacctaatctccacttttttcataggttactacctatacaatgtatgtaatttgtttcccctagcttactgataaacctaatattcaatgaacttccatttgtattcaaatttgtgtcataccagaaagctctacatttgcagatgttcaaatattgtaaaactttggtgcattgttatttaatagctgtgatcagtgattttcaaacctcaaatatagtatattaacaaatt

SEQ ID NO: 4 - DNA, 20 bp, Artificial Sequence
  Description of Combined DNA/RNA Molecule Synthetic siRNA sequence
gaugaaggaggcgcucagtt

SEQ ID NO: 5 - DNA, 21 bp, Artificial Sequence
  Description of Combined DNA/RNA Molecule Synthetic siRNA sequence
ggcugaguucuggcuggactt

SEQ ID NO: 6 - protein, 11 aa, Artificial Sequence
  Description of Artificial Sequence Synthetic peptide
QKRPSQRSKYL